(19) United States
(12) Patent Application Publication
(10) Pub. No.: US 2021/0308034 A1
(43) Pub. Date: Oct. 7, 2021

(54) PREBIOTIC COMPOSITIONS AND METHODS FOR MAINTAINING A HEALTHY SKIN MICROBIOTA

(71) Applicant: KIMBERLY-CLARK WORLDWIDE, INC., Neenah, WI (US)

(72) Inventors: Elizabeth M. Doney, Appleton, WI (US); David W. Koenig, Menasha, WI (US); Jonathan Hofmekler, Sandy Springs, GA (US)

(73) Assignee: KIMBERLY-CLARK WORLDWIDE, INC., Neenah, WI (US)

(21) Appl. No.: 17/056,800

(22) PCT Filed: May 22, 2019
(86) PCT No.: PCT/US19/33434

§ 371 Date: Nov. 19, 2020

Related U.S. Application Data
(60) Provisional application No. 62/678,347, filed on May 31, 2018.

(51) Int. Cl. A61K 8/73; A61Q 19/00; A61K 8/44; A61K 8/362; A61K 8/34; A61K 8/365; A61K 8/60; A61K 8/41; A61L 15/42; A61L 15/28; A61L 15/20

(52) U.S. Cl. CPC A61K 8/738; A61L 2300/232; A61K 8/44; A61K 8/362; A61K 8/345; A61K 8/73; A61K 8/365; A61K 8/60; A61K 8/602; A61K 8/415; A61L 15/42; A61L 15/28; A61L 15/20; A61L 2300/214; A61L 2300/21; A61L 2300/204; A61Q 19/00

(57) ABSTRACT

A composition for providing or maintaining a healthy skin microbiota and methods of providing or maintaining a healthy skin microbiota are disclosed. The composition can be a prebiotic composition that can include a carrier and a skin microbiota balancing agent. In some aspects, the skin microbiota balancing agent can include at least one combination of carbohydrate sources including a first carbohydrate source and a second carbohydrate source. The skin microbiota balancing agent can be configured to provide at least two of the following desired ratios: a first desired ratio of Corynebaderium to Staphylococcus of 1.3, a second desired ratio of Corynebaderium to Micrococcus of 1.4, and a third desired ratio of Staphylococcus to Micrococcus of 1.1. The carbohydrate sources may be D-alanine, D-threonine, L-alanyl-glycine, succinic acid, α-keto-glutaric acid, m-tantaric acid, bromo succinic acid, mucic acid, phenylethyl-amine, inulin, oxalic acid, pectin, Tween 40 etc.

### BACKGROUND OF THE DISCLOSURE

Human skin serves as the primary physical barrier to protect the body from the external environment. This biological interface includes a variety of bacteria and fungi that help to maintain the barrier function the skin provides. The skin is recognized to have a low abundance, but a high diversity of bacteria. This high diversity of bacteria create complex relationships amongst resident bacteria, transient bacteria, the interaction between such bacteria, and the host's skin and immune system. These relationships are important to skin health.

A variety of factors can affect skin health. Intrinsic factors, such as host genetics and skin biochemical properties, as well as extrinsic factors, such as hygiene practices and host environment conditions, can shift the skin microbiota from its typical state to a dysbiotic state, which can lead to skin barrier breakdown and potentially to disease. A healthy skin microbiota is composed of multiple bacterial species living in distinct proportions within a skin niche.

Prebiotics exist that can be beneficial to various bacteria. However, use of some existing prebiotics may lead to unproportioned growth between host skin bacteria, and thus, may disrupt the natural relationships within a natural skin microbiota.

As such, a need currently exists for improved compositions that can support and maintain a healthy balance and proportions of commensal skin bacteria that lead to a well-balanced skin microbiota, as well as methods to maintain such a skin microbiota.

### SUMMARY OF THE DISCLOSURE

It has now been surprisingly discovered that various compositions including skin microbiota balancing agents can provide for a balanced growth of commensal skin bacteria to support a healthy and natural human skin microbiota. These compositions can be well suited for topical administration to the skin of a human subject to help support and maintain a healthy balance in the skin microbiota, and can be delivered to skin through various delivery mechanisms and methods.

Accordingly, in one embodiment a composition for providing or maintaining a healthy skin microbiota can include a carrier. The composition can also include a skin microbiota balancing agent. The skin microbiota balancing agent can include at least one combination of carbohydrate sources including a first carbohydrate source and a second carbohydrate source. The skin microbiota balancing agent can be configured to provide at least two of the following desired ratios: a first desired ratio of Corynebacterium to Staphylococcus, a second desired ratio of Corynebacterium to Micrococcus, and a third desired ratio of Staphylococcus to Micrococcus.

In another embodiment, a composition for providing or maintaining a healthy skin microbiota can include a carrier. The composition can also include a skin microbiota balancing agent. The skin microbiota balancing agent can include at least one combination of carbohydrate sources. The combination of carbohydrate sources can be selected from the group consisting of: D-Alanine+3-Methyl Glucose, D-Alanine+Oxalic Acid, L-Alanyl-Glycine+γ-Cyclodextrin, L-Alanyl-Glycine+Inulin, L-Alanyl-Glycine+Sebacic Acid, L-Alanyl-Glycine+2,3-Butanediol, L-Alanyl-Glycine+3-Hydroxy 2-Butanone, D-Saccharic Acid+Oxalic Acid+L-Alanine, Succinic Acid+Oxalic Acid+L-Alanine, Succinic Acid+Pectin+L-Alanine, Tween 40+Oxalic Acid+L-Alanine, Tween 40+Pectin+L-Alanine, α-Keto-Glutaric Acid+Inulin+Glycyl-L-Aspartic Acid, α-Keto-Glutaric Acid+Sebacic Acid+Glycyl-L-Aspartic Acid, α-Keto-Glutaric Acid+3-Hydroxy-2-Butanone+Glycyl-L-Aspartic Acid, m-Tartaric Acid+Oxalic Acid+L-Alanine, m-Tartaric Acid+Pectin+L-Alanine, Bromo Succinic Acid+γ-Cyclodextrin+D-glucosaminic Acid, Bromo Succinic Acid+γ-Cyclodextrin+Glycyl-L-Aspartic Acid, Bromo Succinic Acid+Inulin+D-glucosaminic Acid, Bromo Succinic Acid+Inulin+Glycyl-L-Aspartic Acid, Bromo Succinic Acid+3-Methyl Glucose+D-glucosaminic Acid, Bromo Succinic Acid+3-Methyl Glucose+Glycyl-L-Aspartic Acid, Bromo Succinic Acid+Sebacic Acid+D-glucosaminic Acid, Bromo Succinic Acid+Sebacic Acid+glycyl-L-Aspartic Acid, Bromo Succinic Acid+2,3-Butanediol+D-glucosaminic Acid, Bromo Succinic Acid+3-Hydroxy-2-Butanone+D-glucosaminic Acid, Bromo Succinic Acid+Pectin+D-Glucosaminic Acid, Bromo Succinic Acid+Pectin+Glycyl-L-Aspartic Acid, Phenylethyl-amine+α-Cyclodextrin+N-Acetyl-D-Galactosamine, Phenylethyl-amine+Turanose+N-Acetyl-D-Galactosamine, Phenylethyl-amine+L-Homoserine+N-Acetyl-D-Galactosamine, Phenylethyl-amine+2,3-Butanediol+N-Acetyl-D-Galactosamine, and Phenylethyl-amine+3-Hydroxy-2-Butanone+N-Acetyl-D-Galactosamine.

In yet another embodiment, a method for providing or maintaining a healthy skin microbiota on a subject can include creating a prebiotic composition configured to maintain at least one of a first desired ratio of Corynebacterium to Staphylococcus, a second desired ratio of Corynebacterium to Micrococcus, and a third desired ratio of Staphylococcus to Micrococcus. The prebiotic composition can include a carrier. The prebiotic composition can also include a skin microbiota balancing agent. The skin microbiota balancing agent can include at least a first carbohydrate source. The first carbohydrate source can be selected to maintain the at least one of the first desired ratio of Corynebacterium to Staphylococcus, the second desired ratio of Corynebacterium to Micrococcus, and the third desired ratio of Staphylococcus to Micrococcus. The method can further include providing instruction to apply the prebiotic composition to the subject.

### BRIEF DESCRIPTION OF DRAWINGS

A full and enabling disclosure thereof, directed to one of ordinary skill in the art, is set forth more particularly in the remainder of the specification, which makes reference to the appended figures in which:

FIG. 1 is a graph showing twelve exemplary carbohydrate sources from Table 3 and the plotted difference between the respective calculated ratio and desired ratio of Corynebacterium to Staphylococcus (C:S), Corynebacterium to Micrococcus (C:M), and Staphylococcus to Micrococcus (S:M) for each of the twelve exemplary carbohydrate sources.

### DEFINITIONS

As used herein, the term “absorbent article” refers to an article which may be placed against or in proximity to the body (i.e., contiguous with the body) of the wearer to absorb and contain various liquid, solid, and semi-solid exudates discharged from the body. It is to be understood that the present disclosure is applicable to various disposable absorbent articles, including, but not limited to, diapers, diaper pants, training pants, youth pants, swim pants, feminine hygiene products, including, but not limited to, menstrual pads or pants, incontinence products, medical garments, surgical pads and bandages, other personal care or health care garments, and the like without departing from the scope of the present disclosure.

As used herein, the term “skin microbiota balancing agent” refers to any carbohydrate source or combination of carbohydrate sources that provides a calculated ratio of commensal skin bacteria within ±0.25 units of a respective desired ratio of such commensal skin bacteria.

### DETAILED DESCRIPTION OF THE DISLOSURE

The present invention is related to compositions and methods useful in supporting and maintaining a healthy skin microbiota for human subjects. The compositions are particularly well suited for topical administration to the skin, and in particular, in areas of dry to semi-moist skin environments. The compositions can be applied directly to the skin, such as through application in the form of a liquid, cream, or spray. Compositions can be alternatively or additionally applied to the skin through a delivery mechanism such as, for example, a wipe substrate or by being applied to an absorbent article that can deliver the composition to the skin.

Targets of desired ratios between various commensal skin bacteria were developed by considering multiple sources providing relative abundance values of various commensal skin bacteria. The areas of the skin that were researched include dry to semi-moist areas of the skin, such as arms, legs, back, buttocks, etc. For example, it has been documented that the relative abundance of Corynebacterium, Staphylococcus, and γ-proteobacteria on moist skin sites (e.g., inside nostril, armpit, inner elbow, between fingers, side of groin, gluteal crease, behind knee, navel, etc.) can range from about 4% to about 65%, from about 8% to about 48%, and from about 1% to about 10%, respectively. The relative abundance of Micrococcus on the scapula has been documented to be about 40%. From this, the following ratios of commensal bacteria were selected: 65% for Corynebacterium, 48% for Staphylococcus, 45% for Micrococcus, and 10% for γ-proteobacteria. These ratios were then normalized to 100% to provide the following values: Corynebacterium 38.69%, Staphylococcus 28.57%, Micrococcus 26.79%, and y-proteobacteria 5.95%. These percentages were then used to calculate the desired ratios of Corynebacterium to Staphylococcus (1.3), Corynebacterium to Micrococcus (1.4), and Staphylococcus to Micrococcus (1.1) in a healthy skin microbiome. These desired ratios were created because the bacteria of Corynebacterium, Staphylococcus, and Micrococcus can provide a significant majority of the commensal bacteria in a healthy dry to semi-moist skin microbiome. As will be discussed further below, the desired ratios of Corynebacterium to Staphylococcus (1.3), Corynebacterium to Micrococcus (1.4), and Staphylococcus to Micrococcus (1.1) were employed in further research to model appropriate carbohydrate sources and combinations of carbohydrate sources that may be beneficial as skin microbiota balancing agents.

Skin Microbiota Balancing Agents

After creating desired ratios of various commensal skin bacteria in a healthy skin microbiome, 188 different carbohydrate sources were screened as the next step in determining potential prospects for skin microbiota balancing agents. The screening process was set up to determine which carbohydrate sources promoted commensal and/or pathogenic bacteria as well as to obtain individual and combinations of carbohydrate sources that could serve as microbiota balancing agents that can support or maintain the healthy proportions of commensal skin bacteria of Corynebacterium, Staphylococcus, and Micrococcus. The screening was conducted utilizing Biolog phenotype microarray plates (Hayward, Calif.). Table 1 provides a coded list of the commensal and pathogenic skin bacteria that the carbohydrates were screened against.

TABLE 1
Bacteria Codes for Carbohydrate Screening

| Code | Bacteria | Type |
|---|---|---|
| A | Corynebacterium minutissimum (ATCC 23348) | Pathogenic |
| B | Corynebacterium jeikum (ATCC 33033) | Commensal |
| C | Klebsiella pneumoniae (ATCC BAA-2146) | Pathogenic |
| D | Micrococcus luteus (ATCC 49732) | Commensal |
| E | Micrococcus luteus (SK58) | Commensal |
| F | Pseudomonas aeruginosa (ATCC 9027) | Pathogenic |
| G | Staphylococcus aureus (ATCC 6538) | Pathogenic |
| H | Staphylococcus aureus (ATCC 25904) (Newman) | Pathogenic |
| I | Staphylococcus epidermidis (ATCC 14990) | Commensal |
| J | Staphylococcus epidermidis (M23864:W2) | Commensal |

Table 2 provides the results of screening various carbohydrate sources against the commensal and pathogenic bacteria codes listed in Table 1. The Bacteria Codes B, D, E, I, and J are commensal bacteria and are shaded in Table 2 for references purposes. The bacteria codes were each placed in a carbon microarray plate and screened through the Carbohydrate Screening Test Method, as described in the Test Methods section herein. If a particular bacteria code utilized a specific carbohydrate source as described in the Carbohydrate Screening Test Method, then the corresponding cell in Table 2 was marked with a Y (short for “Yes”). If a particular bacteria code did not utilize the carbohydrate source, then the corresponding cell in Table 2 was left blank. Many of the carbohydrate sources were utilized by both commensal and pathogenic bacteria, and as such, would not provide an advantage for selective growth of the commensal bacteria. However, some of the carbohydrate sources were only used by commensal bacteria and were identified for further testing as described herein.

TABLE 2
Carbohydrate Screening Results

| | Bacteria Codes | | | | | | | | | |
|---|---|---|---|---|---|---|---|---|---|---|
| Carbohydrates | A | B | C | D | E | F | G | H | I | J |
| L-Arabinose | | | Y | | | Y | | | | |
| N-Acetyl-D- | Y | Y | | | Y | | | | | |
| Glucosamine | | | | | | | | | | |
| D-Saccharic Acid | Y | | | | | | | | | |
| Succinic Acid | Y | | | | | | | | | |
| D-Galactose | | Y | Y | | Y | Y | | | | Y |
| L-Aspartic Acid | | Y | Y | | | | | | | |
| L-Proline | | Y | | | Y | | | | | |
| D-Alanine | | Y | | | Y | | | | | |
| D-Trehalose | | Y | Y | Y | Y | Y | Y | Y | | |
| D-Mannose | Y | Y | Y | | Y | | Y | Y | | |
| Dulcitol | | Y | | | Y | | | | | |
| D-Sorbitol | | | Y | | Y | Y | | | | |
| Glycerol | | Y | Y | | | | | | | Y |
| L-Fucose | | Y | Y | | Y | | | | | |
| D-Glucuronic | | Y | Y | | Y | Y | | | | |
| Acid | | | | | | | | | | |
| D-Gluconic Acid | | Y | Y | | Y | Y | | | | Y |
| D,L-α-Glycerol- | | Y | | | Y | | | | | Y |
| Phosphate | | | | | | | | | | |
| D-Xylose | | | Y | | | Y | | | | |
| L-Lactic Acid | Y | Y | | | Y | | | | Y | |
| Formic Acid | | Y | | | Y | | | | | |
| D-Mannitol | | Y | Y | | Y | Y | Y | Y | | |
| L-Glutamic Acid | | Y | | | | Y | | | | |
| D-Glucose-6- | | | Y | | Y | Y | Y | | Y | Y |
| Phosphate | | | | | | | | | | |
| D-Galactonic | | | Y | | | Y | | | | |
| Acid-γ-Lactone | | | | | | | | | | |
| D,L-Malic Acid | | Y | | | Y | | | | Y | Y |
| L-Rhamnose | | Y | Y | | Y | | | | | |
| D-Fructose | Y | Y | Y | | Y | | Y | Y | | Y |
| Acetic Acid | | Y | | | Y | | | | | |
| α-D-Glucose | Y | Y | Y | | Y | Y | Y | Y | Y | Y |
| Maltose | | Y | Y | | Y | Y | | Y | Y | Y |
| D-Melibiose | | Y | Y | | Y | Y | | | | |
| Thymidine | Y | Y | | | Y | | | | | |
| L-Asparagine | | Y | | | | Y | | | | |
| D-Aspartic Acid | | Y | | | Y | | | | | |
| D-Glucosaminic | | | | | Y | | | | | |
| Acid | | | | | | | | | | |
| 1,2-Propanediol | | Y | | | Y | | | | | |
| Tween 40 | | Y | | | | | | | | |
| α-Keto-Glutaric | | Y | | | | | | | | |
| Acid | | | | | | | | | | |
| α-Keto-Butyric | | Y | | | | | | | | |
| Acid | | | | | | | | | | |
| α-Methyl-D- | | Y | Y | | Y | | | | | |
| Galactoside | | | | | | | | | | |
| α-D-Lactose | | Y | | | Y | | | | Y | Y |
| Lactulose | | Y | | | Y | | | | Y | Y |
| Sucrose | Y | Y | Y | | Y | Y | Y | | | Y |
| Uridine | | Y | Y | Y | | | | | | |
| L-Glutamine | | Y | Y | | | | | | | |
| m-Tartaric Acid | | Y | | | | | | | | |
| D-Glucose-1- | | Y | Y | | Y | | | | | |
| Phosphate | | | | | | | | | | |
| D-Fructose-6- | | Y | | | Y | | Y | Y | | Y |
| Phosphate | | | | | | | | | | |
| Tween 80 | | Y | | | Y | | | | Y | |
| α-Hydroxy | | Y | | | Y | | | | | |
| Glutaric Acid-γ- | | | | | | | | | | |
| Lactone | | | | | | | | | | |
| α-Hydroxy Butyric | | Y | | | | | | | | |
| Acid | | | | | | | | | | |
| β-Methyl-D- | | Y | Y | | Y | Y | | Y | | |
| Glucoside | | | | | | | | | | |
| Adonitol | | Y | Y | | Y | Y | | | | |
| Maltotriose | Y | Y | Y | | Y | Y | | Y | Y | Y |
| Adenosine | | Y | | | Y | | | | | |
| Glycyl-L-Aspartic | | | | | Y | | | | | |
| Acid | | | | | | | | | | |
| Citric Acid | | Y | | | | | | | | |
| m-Inositol | | Y | Y | | Y | | | | | |
| D-Threonine | | Y | | | Y | | | | | |
| Fumaric Acid | | Y | | | Y | | | | | |
| Bromo Succinic | | Y | | | | | | | | |
| Acid | | | | | | | | | | |
| Propionic Acid | | Y | | | Y | | | | | |
| Mucic Acid | | Y | | | | | | | | |
| Glycolic Acid | | Y | | | Y | | | | | |
| Glyoxylic Acid | | Y | | | | | | | | |
| D-Cellobiose | | Y | Y | | Y | | | | Y | |
| Inosine | | Y | Y | | Y | | | Y | | |
| Glycyl-L-Glutamic | | | | | Y | | | | | |
| Acid | | | | | | | | | | |
| Tricarballylic Acid | | | | | Y | | | | | |
| L-Serine | | Y | | | Y | | | | Y | Y |
| L-Threonine | | Y | | | Y | | | | | |
| L-Alanine | | | | | Y | | | | | |
| L-Alanyl-Glycine | | Y | | | Y | | | | | |
| Acetoacetic Acid | | Y | | | Y | | | | | |
| N-Acetyl-β-D- | | Y | | | Y | | | | | |
| Mannosamine | | | | | | | | | | |
| Mono Methyl | | Y | | | Y | | | | Y | |
| Succinate | | | | | | | | | | |
| Methyl Pyruvate | | Y | | | | | | | Y | Y |
| D-Malic Acid | | Y | | | Y | | | | Y | |
| L-Malic Acid | | Y | | | Y | | | | | |
| m-Hydroxy | | | Y | | | | | | | |
| Phenyl Acetic | | | | | | | | | | |
| Acid | | | | | | | | | | |
| Tyramine | | Y | Y | | | Y | | | | |
| D-Psicose | | Y | Y | | Y | Y | | | | |
| Glucuronamide | | Y | | | Y | | | | | |
| Pyruvic Acid | | Y | | Y | | Y | | | Y | Y |
| L-Galactonic | | Y | Y | | | | | | | |
| Acid-γ-Lactone | | | | | | | | | | |
| D-Galacturonic | | Y | | Y | Y | | | | | |
| Acid | | | | | | | | | | |
| Phenylethyl-amine | | Y | | | | | | | | |
| 2-Aminoethanol | | Y | | Y | Y | | | | | |
| Chondroitin | | | | Y | | | | | | |
| Sulfate C | | | | | | | | | | |
| α-Cyclodextrin | | | | | | | | | Y | Y |
| β-Cyclodextrin | | | | Y | | | | | Y | Y |
| γ-Cyclodextrin | | | | | | | | | | Y |
| Dextrin | | | Y | | | | | | | Y |
| Gelatin | | | | Y | | | | | | Y |
| Inulin | | | | | | | | | Y | Y |
| Mannan | | | | | | | | | | Y |
| Pectin | | | | | | | | | Y | Y |
| N-Acetyl-D- | | | | Y | | | | | | |
| Galactosamine | | | | | | | | | | |
| Amygdalin | | | | Y | | | | | | Y |
| D-Arabinose | | | | Y | | | | | | |
| D-Arabitol | | | Y | | | Y | | | | |
| Arbutin | | | Y | | | | | | | |
| 3-0-β-D-Galacto- | | | Y | | | | | | | |
| pyranosyl-D- | | | | | | | | | | |
| Arabinose | | | | | | | | | | |
| Gentiobiose | | | Y | | | Y | | | | |
| Lactitol | | | Y | | | | | | Y | Y |
| D-Melezitose | | | | Y | | | | | Y | |
| Maltitol | | | Y | | | Y | | | | |
| β-Methyl-D- | | | Y | | | Y | | | | |
| Galactoside | | | | | | | | | | |
| 3-Methyl Glucose | | | | | | | | | Y | Y |
| 3-Methyl-D- | | | | Y | | | | | | Y |
| Glucuronic Acid | | | | | | | | | | |
| α-Methyl-D- | | | | Y | | | | | | |
| Mannoside | | | | | | | | | | |
| Palatinose | | | Y | | | Y | | | Y | Y |
| D-Raffinose | | | Y | | | | | | | |
| Salicin | | | Y | Y | | Y | | | | |
| L-Sorbose | | | Y | Y | | Y | | | | |
| Stachyose | | | Y | Y | | Y | | | | |
| Turanose | | | | | | | | | Y | |
| N-Acetyl-D- | | | | | | | | | | Y |
| Glucosaminitol | | | | | | | | | | |
| γ-Amino Butyric | | | | | | | | | Y | Y |
| Acid | | | | | | | | | | |
| δ-Amino Valeric | | | | | | | | | | Y |
| Acid | | | | | | | | | | |
| Butyric Acid | | | | | | Y | | | | |
| Capric Acid | | | | | | Y | | | | |
| Caproic Acid | | | | | | Y | | | | |
| Citraconic Acid | | | | Y | | | | | Y | Y |
| Citramalic Acid | | | | | | Y | | | | |
| D-Glucosamine | | | Y | | | Y | | | | |
| 4-Hydroxy | | | | Y | | | | | | |
| Benzoic Acid | | | | | | | | | | |
| β-Hydroxy Butyric | | | | | | Y | | | Y | |
| Acid | | | | | | | | | | |
| Itaconic Acid | | | | | | Y | | | | |
| D-Lactic Acid | | | Y | | | | | | | |
| Methyl Ester | | | | | | | | | | |
| Malonic Acid | | | Y | Y | | Y | | | | |
| Melibionic Acid | | | Y | | | | | | | |
| Oxalic Acid | | | | | | | | | Y | Y |
| Oxalomalic Acid | | | | Y | | | | | | Y |
| Quinic Acid | | | Y | | | Y | | | | |
| D-Ribono-1,4- | | | | Y | | | | | | |
| Lactone | | | | | | | | | | |
| Sebacic Acid | | | | | | | | | Y | Y |
| Succinamic Acid | | | | Y | | | | | Y | |
| L-Tartaric Acid | | | Y | | | Y | | | | |
| Acetamide | Y | | | | | | | | | |
| L-Alaninamide | | | | Y | | | | | | Y |
| N-Acetyl-L- | | | | Y | | Y | | | | |
| Glutamic Acid | | | | | | | | | | |
| L-Arginine | | | | Y | | Y | | | | |
| L-Histidine | | | Y | | | Y | | | | |
| L-Homoserine | | | | | | | | | Y | |
| L-Isoleucine | | | | | | | | | | |
| L-Leucine | | | | Y | | | | | | |
| L-Methionine | Y | | | | | | | | | |
| L-Ornithine | | | | | | Y | | | | |
| L-Pyroglutamic | | | | | | Y | | | | |
| Acid | | | | | | | | | | |
| D,L-Carnitine | | | | | | Y | | | | Y |
| D.L-Octopamine | | | Y | | | Y | | | | Y |
| Putrescine | | | Y | | | | | | Y | Y |
| Dihydroxy | | | Y | | | | | | | |
| Acetone | | | | | | | | | | |
| 2,3-Butanediol | | | | | | | | | Y | Y |
| 3-Hydroxy 2- | | | | | | | | | Y | Y |
| Butanone | | | | | | | | | | |

As shown in the results from Table 2, 154 different carbohydrate sources were utilized by at least one of the bacteria codes tested. Of these 154 different carbohydrate sources, 79 were utilized only by commensal bacteria and 27 were only utilized by pathogenic bacteria. Additionally, 34 additional carbohydrate sources were tested, but were not utilized by any of the bacteria, and thus, were not listed in Table 2. The carbohydrate sources that were not utilized by any of the bacteria codes tested were: D-Serine, D-Ribose, Tween 20, 2-Deoxy Adenosine, Glycyl-L-Proline, p-hydroxy phenyl acetic acid, L-Lyxose, Glycogen, Laminarin, N-Acetyl-Neuraminic Acid, β-D-Allose, L-Arabitol, 2-Deoxy-D-Ribose, i-Erythritol, D-Fucose, L-Glucose, α-Methyl-D-Glucoside, β-Methyl-D-Xyloside, Sedoheptulosan, D-Tagatose, Xylitol, 2-Hydroxy Benzoic Acid, y-Hydroxy Butyric Acid, α-Keto-Valeric Acid, 5-Keto-D-Gluconic Acid, Sorbic Acid, D-Tartaric Acid, Glycine, Hydroxy-L-Proline, L-Lysine, L-Phenylalanine, L-Valine, Sec-Butylamine, and 2,3-Butanone.

For the 79 carbohydrate sources that were only utilized by the commensal bacteria, further analysis was conducted to identify skin microbiota balancing agents. To do so, the respective microarray data for the 79 carbohydrate sources was run through the GrowthRates 2.1 program to obtain growth parameters, such as growth rates and maximum yield data as described by the Belingham Research Institute, Hall, B. G., H. Acar and M. Barlow. 2014 Growth Rates Made Easy. Mol. Biol. Evol. 31:232-238 doi:10.1093/molbev/mst197. The max yield parameters generated were utilized to calculate ratios of tested commensal bacteria. The ratios calculated were compared back to the desired ratios identified above (Corynebacterium to Staphylococcus of 1.3, Corynebacterium to Micrococcus of 1.4, and Staphylococcus to Micrococcus of 1.1). The results of this methodology are shown in Table 3, with the ratios between various bacteria genera mentioned above being abbreviated for formatting purposes, such that Corynebacterium to Staphylococcus is listed as C:S, Corynebacterium to Micrococcus is listed as C:M, and Staphylococcus to Micrococcus is listed as S:M. As described in the definitions section herein, a skin microbiota balancing agent is any carbohydrate source or combination of carbohydrate sources that provides a calculated ratio of commensal bacteria within ±0.25 units of a respective desired ratio of such commensal bacteria. For reference purposes, the cells in Table 3 that include a difference calculation of a respective commensal bacteria ratio to be within ±0.25 have been highlighted.

As an example of this methodology used to calculate the commensal bacteria ratios and the difference between the calculated ratios and the desired ratios, an example code from Table 3 will be described. C. jeikeium and M. luteus SK58 reached max yields of 0.157 and 0.098 respectively when grown in the presence of D-alanine. S. epidermidis M23864:W2 reached a max yield of 0.123 in the presence of 3-methyl glucose. Thus, it was expected that if D-alanine and 3-methy glucose were mixed together (Code No. 12 in Table 3), the mixture should support the growth of all three commensal bacteria. The max yields were then used to calculate ratios the bacteria would reach in a co-culture. The calculated ratios for this example, and as shown in Table 3, are as follows: Corynebacterium to Staphylococcus (1.28), Corynebacterium to Micrococcus (1.60), and Staphylococcus to Micrococcus (1.26). The difference between the desired ratios and each respective calculated ratios is at most ±0.20, as the difference between the desired ratio of Corynebacterium to Staphylococcus of 1.3 and the calculated ratio of Corynebacterium to Staphylococcus of 1.28 is 0.02, the difference between the desired ratio of Corynebacterium to Micrococcus of 1.4 and the calculated ratio of Corynebacterium to Micrococcus of 1.6 is -0.20, and the difference between the desired ratio of Staphylococcus to Micrococcus of 1.1 and the calculated ratio of Staphylococcus to Micrococcus of 1.26 is -0.16. Thus, the combination of D-Alanine+3-Methyl Glucose (Code No. 12) was a preferred combination of carbohydrate sources because it provided all three of the desired ratios of commensal bacteria of Corynebacterium to Staphylococcus of 1.3, Corynebacterium to Micrococcus of 1.4, and Staphylococcus to Micrococcus of 1.1 by having each of the calculated ratios be within ±0.25 units from the respective desired ratio.

As can be seen from Table 3, the methodology involved calculating the ratios for Corynebacterium to Staphylococcus, Corynebacterium to Micrococcus, and Staphylococcus to Micrococcus for 2532 unique carbohydrate sources that included one or more carbohydrates. As discussed above, a skin microbiota balancing agent can be considered as any carbohydrate source or combination of carbohydrate sources that provided at least one of the desired ratios of Corynebacterium to Staphylococcus, Corynebacterium to Micrococcus, and Staphylococcus to Micrococcus by providing a calculated ratio of commensal bacteria within ±0.25 of the respective desired ratio of commensal bacteria (Corynebacterium to Staphylococcus of 1.3, Corynebacterium to Micrococcus of 1.4, and Staphylococcus to Micrococcus of 1.1). Due to the screening process discussed above, a large number of the carbohydrate sources analyzed in Table 3 provided at least one of the desired ratios, and thus, could serve as a skin microbiota balancing agent. However, a much smaller number of carbohydrate sources tested provided two or more of the desired ratios, or even more preferably, all three of the desired ratios.

Reviewing the results in Table 3, only 119 of the 2532 tested carbohydrate combinations provided two or more of the desired ratios of Corynebacterium to Staphylococcus, Corynebacterium to Micrococcus, and Staphylococcus to Micrococcus. These skin microbiota balancing agents that provided two or three of the desired ratios of Corynebacterium to Staphylococcus, Corynebacterium to Micrococcus, and Staphylococcus to Micrococcus were: D-Alanine+γ-Cyclodextrin, D-Alanine+Sebacic Acid, D-Alanine+2,3-Butanediol, D-Aspartic Acid+Oxalic Acid, D-Aspartic Acid+Pectin, D-Threonine+3-Methyl Glucose, D-Threonine+Pectin, Fumaric Acid+α-Cyclodextrin, Fumaric Acid+Mannan, L-Alanyl-Glycine+3-Methyl Glucose, L-Alanyl-Glycine+Oxalic Acid, L-Alanyl-Glycine+Pectin, L-Malic Acid+Mannan, D-Saccharic Acid+Pectin+L-Alanine, Succinic Acid+3-Methyl Glucose+D-Glucosaminic Acid, Succinic Acid+3-Methyl Glucose+L-Alanine, Succinic Acid+3-Methyl Glucose+Glycyl-L-Aspartic Acid, Succinic Acid+Pectin+D-Glucosaminic Acid, Succinic Acid+Pectin+Glycyl-L-Aspartic Acid, Tween 40+3-Methyl Glucose+D-glucosaminic Acid, Tween 40+3-Methyl Glucose+L-Alanine, Tween 40+3-Methyl Glucose+Glycyl-L-Aspartic Acid, Tween 40+Pectin+D-glucosaminic Acid, Tween 40+Pectin+Glycyl-L-Aspartic Acid, α-Keto-Glutaric Acid+γ-Cyclodextrin+Glycyl-L-Aspartic Acid, α-Keto-Glutaric Acid+Inulin+D-glucosaminic Acid, α-Keto-Glutaric Acid+3-Methyl Glucose+Glycyl-L-Aspartic Acid, α-Keto-Glutaric Acid+γ-Amino Butyric Acid+Glycyl-L-Aspartic Acid, α-Keto-Glutaric Acid+Sebacic Acid+D-glucosaminic Acid, α-Keto-Glutaric Acid+2,3-Butanediol+Glycyl-L-Aspartic Acid, α-Keto-Glutaric Acid+3-Hydroxy-2-Butanone+D-glucosaminic Acid, α-Keto-Butyric Acid+α-Cyclodextrin+N-Acetyl-D-Galactosamine, α-Keto-Butyric Acid+Inulin+D-glucosaminic Acid, α-Keto-Butyric Acid+Inulin+Glycyl-L-Aspartic Acid, α-Keto-Butyric Acid+3-Methyl Glucose+D-glucosaminic Acid, α-Keto-Butyric Acid+3-Methyl Glucose+Glycyl-L-Aspartic Acid, α-Keto-Butyric Acid+Oxalic Acid+L-Alanine, α-Keto-Butyric Acid+Sebacic Acid+D-glucosaminic Acid, α-Keto-Butyric Acid+Sebacic Acid+Glycyl-L-Aspartic Acid, α-Keto-Butyric Acid+L-Homoserine+N-Acetyl-D-Galactosamine, α-Keto-Butyric Acid+2,3-Butanediol+N-Acetyl-D-Galactosamine, α-Keto-Butyric Acid+3-Hydroxy-2-Butanone+N-Acetyl-D-Galactosamine, m-Tartaric Acid+Pectin+D-Glucosaminic Acid, m-Tartaric Acid+Pectin+Glycyl-L-Aspartic Acid, α-Hydroxy Butyric Acid+Inulin+D-glucosaminic Acid, α-Hydroxy Butyric Acid+Inulin+Glycyl-L-Aspartic Acid, α-Hydroxy Butyric Acid+3-Methyl Glucose+D-glucosaminic Acid, α-Hydroxy Butyric Acid+3-Methyl Glucose+Glycyl-L-Aspartic Acid, α-Hydroxy Butyric Acid+Oxalic Acid+L-Alanine, α-Hydroxy Butyric Acid+Sebacic Acid+D-glucosaminic Acid, α-Hydroxy Butyric Acid+Sebacic Acid+Glycyl-L-Aspartic Acid, α-Hydroxy Butyric Acid+2,3-Butanediol+N-Acetyl-D-Galactosamine, α-Hydroxy Butyric Acid+3-Hydroxy-2-Butanone+N-Acetyl-D-Galactosamine, Citric Acid+α-Cyclodextrin+Glycyl-L-Glutamic Acid, Citric Acid+α-Cyclodextrin+Tricarballylic Acid, Citric Acid+Mannan+Glycyl-L-Glutamic Acid, Citric Acid+Mannan+Tricarballylic Acid, Citric Acid+Oxalic Acid+L-Alanine, Citric Acid+Pectin+L-Alanine, Bromo Succinic Acid+2,3-Butanediol+N-Acetyl-D-Galactosamine, Bromo Succinic Acid+2,3-Butanediol+glycyl-L-Aspartic Acid, Bromo Succinic Acid+3-Hydroxy-2-Butanone+N-Acetyl-D-Galactosamine, Bromo Succinic Acid+3-Hydroxy-2-Butanone+Glycyl-L-Aspartic Acid, Bromo Succinic Acid+Pectin+L-Alanine, Mucic Acid+Inulin+D-glucosaminic Acid, Mucic Acid+Inulin+Glycyl-L-Aspartic Acid, Mucic Acid+3-Methyl Glucose+D-glucosaminic Acid, Mucic Acid+3-Methyl Glucose+Glycyl-L-Aspartic Acid, Mucic Acid+Oxalic Acid+L-Alanine, Mucic Acid+Sebacic Acid+D-glucosaminic Acid, Mucic Acid+Sebacic Acid+Glycyl-L-Aspartic Acid, Mucic Acid+2,3-Butanediol+N-Acetyl-D-Galactosamine, Mucic Acid+3-Hydroxy-2-Butanone+N-Acetyl-D-Galactosamine, Glyocylic Acid+Inulin+Glycyl-L-Aspartic Acid, Glyocylic Acid+3-Methyl Glucose+D-glucosaminic Acid, Glyocylic Acid+3-Methyl Glucose+Glycyl-L-Aspartic Acid, Glyocylic Acid+Oxalic Acid+L-Alanine, Glyocylic Acid+Sebacic Acid+D-glucosaminic Acid, Glyocylic Acid+Sebacic Acid+Glycyl-L-Aspartic Acid, Glyocylic Acid+2,3-Butanediol+N-Acetyl-D-Galactosamine, Glyocylic Acid+3-Hydroxy-2-Butanone+N-Acetyl-D-Galactosamine, Phenylethyl-amine+3-Methyl Glucose+D-glucosaminic Acid, Phenylethyl-amine+3-Methyl Glucose+N-Acetyl-D-Galactosamine, Phenylethyl-amine+3-Methyl Glucose+Glycyl-L-Aspartic Acid, Phenylethyl-amine+Oxalic Acid+L-Alanine, Phenylethyl-amine+Oxalic Acid+N-Acetyl-D-Galactosamine, Phenylethyl-amine+Pectin+N-Acetyl-D-Galactosamine, D-Alanine+3-Methyl Glucose, D-Alanine+Oxalic Acid, L-Alanyl-Glycine+γ-Cyclodextrin, L-Alanyl-Glycine+Inulin, L-Alanyl-Glycine+Sebacic Acid, L-Alanyl-Glycine+2,3-Butanediol, L-Alanyl-Glycine+3-Hydroxy 2-Butanone, D-Saccharic Acid+Oxalic Acid+L-Alanine, Succinic Acid+Oxalic Acid+L-Alanine, Succinic Acid+Pectin+L-Alanine, Tween 40+Oxalic Acid+L-Alanine, Tween 40+Pectin+L-Alanine, α-Keto-Glutaric Acid+Inulin+Glycyl-L-Aspartic Acid, α-Keto-Glutaric Acid+Sebacic Acid+Glycyl-L-Aspartic Acid, α-Keto-Glutaric Acid+3-Hydroxy-2-Butanone+Glycyl-L-Aspartic Acid, m-Tartaric Acid+Oxalic Acid+L-Alanine, m-Tartaric Acid+Pectin+L-Alanine, Bromo Succinic Acid+y-Cyclodextrin+D-glucosaminic Acid, Bromo Succinic Acid+γ-Cyclodextrin+Glycyl-L-Aspartic Acid, Bromo Succinic Acid+Inulin+D-glucosaminic Acid, Bromo Succinic Acid+Inulin+Glycyl-L-Aspartic Acid, Bromo Succinic Acid+3-Methyl Glucose+D-glucosaminic Acid, Bromo Succinic Acid+3-Methyl Glucose+Glycyl-L-Aspartic Acid, Bromo Succinic Acid+Sebacic Acid+D-glucosaminic Acid, Bromo Succinic Acid+Sebacic Acid+glycyl-L-Aspartic Acid, Bromo Succinic Acid+2,3-Butanediol+D-glucosaminic Acid, Bromo Succinic Acid+3-Hydroxy-2-Butanone+D-glucosaminic Acid, Bromo Succinic Acid+Pectin+D-Glucosaminic Acid, Bromo Succinic Acid+Pectin+Glycyl-L-Aspartic Acid, Phenylethyl-amine+a-Cyclodextrin+N-Acetyl-D-Galactosamine, Phenylethyl-amine+Turanose+N-Acetyl-D-Galactosamine, Phenylethyl-amine+L-Homoserine+N-Acetyl-D-Galactosamine, Phenylethyl-amine+2,3-Butanediol+N-Acetyl-D-Galactosamine, and Phenylethyl-amine+3-Hydroxy-2-Butanone+N-Acetyl-D-Galactosamine.

Table 3 also documents that only 32 carbohydrate source combinations of the 2352 test combinations provided all three of the desired ratios of Corynebacterium to Staphylococcus, Corynebacterium to Micrococcus, and Staphylococcus to Micrococcus. These skin microbiota balancing agents are even more preferable because they provide a more complete skin microbiota balancing agent by supporting or maintaining three distinct ratios of commensal bacteria within a healthy skin microbiota. These skin microbiota balancing agents were: D-Alanine+3-Methyl Glucose, D-Alanine+Oxalic Acid, L-Alanyl-Glycine+γ-Cyclodextrin, L-Alanyl-Glycine+Inulin, L-Alanyl-Glycine+Sebacic Acid, L-Alanyl-Glycine+2,3-Butanediol, L-Alanyl-Glycine+3-Hydroxy 2-Butanone, D-Saccharic Acid+Oxalic Acid+L-Alanine, Succinic Acid+Oxalic Acid+L-Alanine, Succinic Acid+Pectin+L-Alanine, Tween 40+Oxalic Acid+L-Alanine, Tween 40+Pectin+L-Alanine, α-Keto-Glutaric Acid+Inulin+Glycyl-L-Aspartic Acid, α-Keto-Glutaric Acid+Sebacic Acid+Glycyl-L-Aspartic Acid, α-Keto-Glutaric Acid+3-Hydroxy-2-Butanone+Glycyl-L-Aspartic Acid, m-Tartaric Acid+Oxalic Acid+L-Alanine, m-Tartaric Acid+Pectin+L-Alanine, Bromo Succinic Acid+y-Cyclodextrin+D-glucosaminic Acid, Bromo Succinic Acid+γ-Cyclodextrin+Glycyl-L-Aspartic Acid, Bromo Succinic Acid+Inulin+D-glucosaminic Acid, Bromo Succinic Acid+Inulin+Glycyl-L-Aspartic Acid, Bromo Succinic Acid+3-Methyl Glucose+D-glucosaminic Acid, Bromo Succinic Acid+3-Methyl Glucose+Glycyl-L-Aspartic Acid, Bromo Succinic Acid+Sebacic Acid+D-glucosaminic Acid, Bromo Succinic Acid+Sebacic Acid+glycyl-L-Aspartic Acid, Bromo Succinic Acid+2,3-Butanediol+D-glucosaminic Acid, Bromo Succinic Acid+3-Hydroxy-2-Butanone+D-glucosaminic Acid, Bromo Succinic Acid+Pectin+D-Glucosaminic Acid, Bromo Succinic Acid+Pectin+Glycyl-L-Aspartic Acid, Phenylethyl-amine+α-Cyclodextrin+N-Acetyl-D-Galactosamine, Phenylethyl-amine+Turanose+N-Acetyl-D-Galactosamine, Phenylethyl-amine+L-Homoserine+N-Acetyl-D-Galactosamine, Phenylethyl-amine+2,3-Butanediol+N-Acetyl-D-Galactosamine, and Phenylethyl-amine+3-Hydroxy-2-Butanone+N-Acetyl-D-Galactosamine.

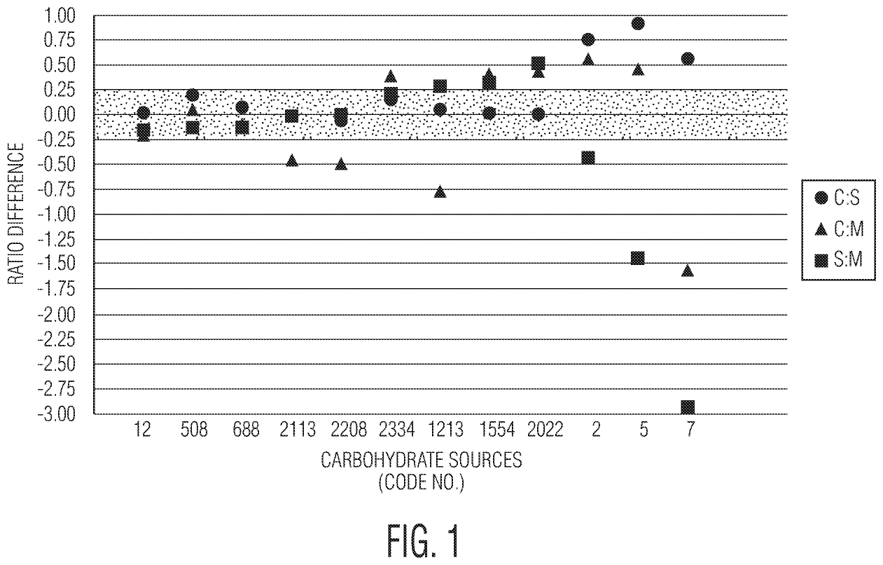

FIG. 1 provides a sample set of data from the experimental analysis described above and documented in Table 3 and provides a visual indication of a sample set of Codes from Table 3 that provide skin microbiota balancing agents, as well as some Codes from Table 3 that do not provide skin microbiota balancing agents. As illustrated in FIG. 1, twelve codes from Table 3 are shown across the horizontal axis with their respective difference ratios between the calculated and desired ratios for Corynebacterium to Staphylococcus (C:S), Corynebacterium to Micrococcus (C:M), and Staphylococcus to Micrococcus (S:M) being shown on the vertical axis. The skin microbiota balancing agents are those carbohydrate sources that provided at least one of the desired ratios described above, or in other words, had at least one ratio difference that was within ±0.25 of the respective desired ratio.

Some of the carbohydrate sources did not provide any of the desired ratios of Corynebacterium to Staphylococcus (C:S), Corynebacterium to Micrococcus (C:M), and Staphylococcus to Micrococcus (S:M), and thus, did not serve as skin microbiota balancing agents. For example, Code Nos. 2, 5, and 7 from Table 3 are illustrated in FIG. 1 as not being able to provide any of the three desired ratios. Reviewing Table 3 shows that a significant number of the codes tested did not provide any of the three desired ratios, and thus, would not suffice as skin microbiota balancing agents.

As described above, some of the carbohydrate sources provided at least one desired ratio of Corynebacterium to Staphylococcus (C:S), Corynebacterium to Micrococcus (C:M), and Staphylococcus to Micrococcus (S:M). Exemplary Code Nos. 12, 508, 688, 2113, 2208, 2334, 1213, 1554, and 2022 from Table 3 are illustrated in FIG. 1 and provide such a result. Reviewing Table 3 shows that several hundred codes of carbohydrate sources of the 2532 codes tested provided at least one desired ratio, and thus, could serve as a skin microbiota balancing agent.

However, only 119 of the 2532 tested carbohydrate sources provided two or more of the desired ratios of Corynebacterium to Staphylococcus, Corynebacterium to Micrococcus, and Staphylococcus to Micrococcus. This represents only 4.7% of the tested carbohydrate sources providing such a result. Exemplary Code Nos. 12, 508, 688, 2113, 2208, and 2334 from Table 3 illustrate such a result in FIG. 1. The carbohydrate sources that provide at least two of the desired ratios are more preferable than the carbohydrate sources providing none or only one of the desired ratios.

Even more unexpected was the result of a carbohydrate source providing all three of the desired ratios of Corynebacterium to Staphylococcus, Corynebacterium to Micrococcus, and Staphylococcus to Micrococcus codes. Exemplary Code Nos. 12, 508, and 688 from Table 3 illustrate such an unexpected result in FIG. 1. The carbohydrate sources from Table 3 that provide all of the desired ratios (and as shown by the exemplary Code Nos. 12, 508, and 688 in FIG. 1) are more preferable than the carbohydrate sources providing none, only one, or even two of the desired ratios. Such combinations of carbohydrates only made up 32 specific codes from the 2532 tested, and thus, represent only about 1.3% of the codes tested.

The composition can be in a wide variety of forms such as, for example, simple solutions (water-based or oil-based), solid forms (e.g. gels or sticks), lotions, suspensions, creams, milks, salves, ointments, sprays, emulsions, oils resins, aerosols, and the like. Preferably, the compositions useful in the present disclosure are soluble to facilitate their formulation for administration to a user.

Carrier

The compositions can include a carrier. The carrier can be any dermatologically acceptable carrier. As used herein, “dermatologically acceptable carrier” generally refers to a carrier that is suitable for topical application to the skin and is compatible with a skin microbiota balancing agent. Liquid carrier materials suitable for use in the instant disclosure include those well-known for use in the cosmetic, pharmaceutical, and medical arts as a basis for ointments, lotions, creams, salves, aerosols, gels, suspensions, sprays, foams, washes, and the like, and may be used in their established levels. In some embodiments, the carrier can comprise from about 0.01% to about 99.98% (by total weight of the composition), depending on the carrier used.

Preferable carrier materials include polar solvent materials, such as water. Other potential carriers include emollients, humectants, polyols, surfactants, esters, perfluorocarbons, silicones, and other pharmaceutically acceptable carrier materials. In one embodiment, the carrier is volatile, allowing for immediate deposition of the antimicrobial ingredient to the desired surface while improving overall usage experience of the product by reducing drying time. Non-limiting examples of these volatile carriers include 5c5t Dimethicone, Cyclomethicone, Methyl Perfluoroisobutyl Ether, Methyl Perfluorobutyl Ether, Ethyl Perfluoroisobutyl Ether and Ethyl Perfluorobutyl Ether.

Where the composition forms a wetting composition, such as described below for use with a wet wipe, the composition will typically include water. The compositions can suitably comprise water in an amount of from about 0.01% (by total weight of the composition) to about 99.98% (by total weight of the composition), or from about 1.00% (by total weight of the composition) to about 99.98% (by total weight of the composition), or from about 50.00% (by total weight of the composition) to about 99.98% (by total weight of the composition), or from about 75.00% (by total weight of the composition) to about 99.98% (by total weight of the composition). In some embodiments, water can comprise an amount from about 50.00% (by total weight of the composition) to about 70.00% (by total weight of the composition). In some embodiments, water can comprise an amount greater than 90.00% (by total weight of the composition).

Emollients

In one embodiment, the compositions can optionally include one or more emollients, which typically act to soften, soothe, and otherwise lubricate and/or moisturize the skin. Suitable emollients that can be incorporated into the compositions include oils such as alkyl dimethicones, alkyl methicones, alkyldimethicone copolyols, phenyl silicones, alkyl trimethylsilanes, dimethicone, dimethicone crosspolymers, cyclomethicone, lanolin and its derivatives, fatty esters, fatty acids, glycerol esters and derivatives, propylene glycol esters and derivatives, alkoxylated carboxylic acids, alkoxylated alcohols, fatty alcohols, and combinations thereof.

Some embodiments of the compositions may include one or more emollients in an amount of from about 0.01% (by total weight of the composition) to about 20% (by total weight of the composition), or from about 0.05% (by total weight of the composition) to about 10% (by total weight of the composition), or from about 0.10% (by total weight of the composition) to about 5% (by total weight of the composition).

Esters

In some embodiments, the compositions include one or more esters. The esters may be selected from cetyl palmitate, stearyl palmitate, cetyl stearate, isopropyl laurate, isopropyl myristate, isopropyl palmitate, and combinations thereof. The fatty alcohols include octyldodecanol, lauryl, myristyl, cetyl, stearyl, behenyl alcohol, and combinations thereof. The fatty acids can include, but are not limited to, capric acid, undecylenic acid, lauric acid, Myristic acid, palmitic acid, stearic acid, oleic acid, linoleic acid, arachidic acid, and behenic acid. Ethers such as eucalyptol, ceteraryl glucoside, dimethyl isosorbic polyglyceryl-3 cetyl ether, polyglyceryl-3 decyltetradecanol, propylene glycol myristyl ether, and combinations thereof can also suitably be used as emollients. Other suitable ester compounds for use in the antimicrobial compositions or the present disclosure are listed in the International Cosmetic Ingredient Dictionary and Handbook, 11th Edition, CTFA, (January, 2006) ISBN-10: 1882621360, ISBN-13: 978-1882621361, and in the 2007 Cosmetic Bench Reference, Allured Pub. Corporation (Jul. 15, 2007) ISBN-10: 1932633278, ISBN-13: 978-1932633276, both of which are incorporated by reference herein to the extent they are consistent herewith.

Humectants

Humectants that are suitable as carriers in the compositions of the present disclosure include, for example, glycerin, glycerin derivatives, hyaluronic acid, hyaluronic acid derivatives, betaine, betaine derivatives, amino acids, amino acid derivatives, glycosaminoglycans, glycols, polyols, sugars, sugar alcohols, hydrogenated starch hydrolysates, hydroxy acids, hydroxy acid derivatives, salts of PCA and the like, and combinations thereof. Specific examples of suitable humectants include honey, sorbitol, hyaluronic acid, sodium hyaluronate, betaine, lactic acid, citric acid, sodium citrate, glycolic acid, sodium glycolate ,sodium lactate, urea, propylene glycol, butylene glycol, pentylene glycol, ethoxydiglycol, methyl gluceth-10, methyl gluceth-20, polyethylene glycols (as listed in the International Cosmetic Ingredient Dictionary and Handbook such as PEG-2 through PEG 10), propanediol, xylitol, maltitol, or combinations thereof.

The compositions of the disclosure may include one or more humectants in an amount of about 0.01% (by total weight of the composition) to about 20% (by total weight of the composition), or about

WO 2019/231776 PCT/US2019/033434 0.05% (by total weight of the composition) to about 10% by total weight of the composition), or about 0.1% (by total weight of the composition) to about 5.0% (by total weight of the composition).

Surfactants

In some embodiments, the composition can include one or more surfactants. In an embodiment where the composition is included in a wipe, the composition may also likely include one or more surfactants. These may be selected from anionic, cationic, nonionic, zwitterionic, and amphoteric surfactants. Amounts of surfactants may range from 0.01 to 30%, or from 10 to 30%, or from 0.05 to 20%, or from 0.10 to 15% by total weight of the composition. In some embodiments, such as when the wetting composition is used with a wipe, the surfactant can comprise less than 5% by total weight of the wetting composition.

Suitable anionic surfactants include, but are not limited to, C₈ to C₂₂ alkane sulfates, ether sulfates and sulfonates. Among the suitable sulfonates are primary C₈ to C₂₂ alkane sulfonate, primary C₈ to C₂₂ alkane disulfonate, C₈ to C₂₂ alkene sulfonate, C₈ to C₂₂ hydroxyalkane sulfonate or alkyl glyceryl ether sulfonate. Specific examples of anionic surfactants include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium laureth sulfate, sodium lauryl sarcosinate, sodium lauroyl sarcosinate, potassium lauryl sulfate, sodium trideceth sulfate, sodium methyl lauroyl taurate, sodium lauroyl isethionate, sodium laureth sulfosuccinate, sodium lauroyl sulfosuccinate, sodium tridecyl benzene sulfonate, sodium dodecyl benzene sulfonate, sodium lauryl amphoacetate and mixtures thereof. Other anionic surfactants include the C₈ to C₂₂ acyl glycinate salts. Suitable glycinate salts include sodium cocoylglycinate, potassium cocoylglycinate, sodium lauroylglycinate, potassium lauroylglycinate, sodium myristoylglycinate, potassium myristoylglycinate, sodium palmitoylglycinate, potassium palmitoylglycinate, sodium stearoylglycinate, potassium stearoylglycinate, ammonium cocoylglycinate and mixtures thereof. Cationic counter-ions to form the salt of the glycinate may be selected from sodium, potassium, ammonium, alkanolammonium and mixtures of these cations.

Suitable cationic surfactants include, but are not limited to alkyl dimethylamines, alkyl amidopropylamines, alkyl imidazoline derivatives, quaternised amine ethoxylates, and quaternary ammonium compounds.

Suitable nonionic surfactants include, but are not limited to, alcohols, acids, amides or alkyl phenols reacted with alkylene oxides, especially ethylene oxide either alone or with propylene oxide.

Specific nonionics are C₆ to C₂₂ alkyl phenols-ethylene oxide condensates, the condensation products of C₈ to C₁₃ aliphatic primary or secondary linear or branched alcohols with ethylene oxide, and products made by condensation of ethylene oxide with the reaction products of propylene oxide and ethylenediamine. Other nonionics include long chain tertiary amine oxides, long chain tertiary phosphine oxides and dialkyl sulphoxides, alkyl polysaccharides, amine oxides, block copolymers, castor oil ethoxylates, ceto-oleyl alcohol ethoxylates, ceto-stearyl alcohol ethoxylates, decyl alcohol ethoxylates, dinonyl phenol ethoxylates, dodecyl phenol ethoxylates, end-capped ethoxylates, ether amine derivatives, ethoxylated alkanolamides, ethylene glycol esters, fatty acid alkanolamides, fatty alcohol alkoxylates, lauryl alcohol ethoxylates, mono-branched alcohol ethoxylates, natural alcohol ethoxylates, nonyl phenol ethoxylates, octyl phenol ethoxylates, oleyl amine ethoxylates, random copolymer alkoxylates, sorbitan ester ethoxylates, stearic acid ethoxylates, stearyl amine ethoxylates, synthetic alcohol ethoxylates, tall oil fatty acid ethoxylates, tallow amine ethoxylates and trid tridecanol ethoxylates.

Suitable zwitterionic surfactants include, for example, alkyl amine oxides, alkyl hydroxysultaines, silicone amine oxides, and combinations thereof. Specific examples of suitable zwitterionic surfactants include, for example, 4-[N,N-di(2-hydroxyethyl)-N-octadecylammonio]-butane-1-carboxylate, S-[S-3-hydroxypropyl-S-hexadecylsulfonio]-3-hydroxypentane-1-sulfate, 3-[P,P-diethyl-P-3,6,9-trioxatetradexopcylphosphonio]-2-hydroxypropane-1-phosphate, 3-[N,N-dipropyl-N-3-dodecoxy-2-hydroxypropylammonio]-propane-1-phosphonate, 3-(N,N-dimethyl-N-hexadecylammonio)propane-1-sulfonate, 3-(N ,N-dimethyl-N-hexadecylammonio)-2-hydroxypropane-1 -sulfonate, 4-[N,N-di(2-hydroxyethyl)-N-(2-hydroxydodecyl)ammonio]-butane-1-carboxylate, 3-[S-ethyl-S-(3-dodecoxy-2-hydroxypropyl)sulfonio]-propane-1-phosphate, 3-[P,P-dimethyl-P-dodecylphosphonio]-propane-1-phosphonate, 5-[N,N-di(3-hydroxypropyl)-N-hexadecylammonio]-2-hydroxy-pentane-1-sulfate, lauryl hydroxysultaine and combinations thereof.

Suitable amphoteric surfactants include, but are not limited to, derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one substituent contains an anionic group, e.g., carboxy, sulfonate, sulfate, phosphate, or phosphonate. Illustrative amnphoterics are coco dimethyl carboxymethyl betaine, cocoamidopropyl betaine, cocobetaine, oleyl betaine, cetyl dimethyl carboxymethyl betaine, lauryl bis-(2-hydroxyethyl)carboxymethyl betaine, stearyl bis-(2-hydroxypropyl)carboxymethyl betaine, oleyl dimethyl gamma-carboxypropyl betaine, lauryl bis-(2-hydroxypropyl)alpha-carboxyethyl betaine, cocoamphoacetates, and combinations thereof. The sulfobetaines may include stearyl dimethyl sulfopropyl betaine, lauryl dimethyl sulfoethyl betaine, lauryl bis-(2-hydroxyethyl)sulfopropyl betaine and combinations thereof.

Rheology Modifiers

Optionally, one or more rheology modifiers, such as thickeners, may be added to the composition. Suitable rheology modifiers are compatible with the skin microbiota balancing agent. As used herein, “compatible” refers to a compound that, when mixed with the skin microbiota balancing agent, does not adversely affect the properties of the skin microbiota balancing agent.

A thickening system is used in the compositions to adjust the viscosity and stability of the compositions. Specifically, thickening systems prevent the composition from running off of the hands or body during dispensing and use of the composition. When the composition is used with a wipe product, a thicker formulation can be used to prevent the composition from migrating from the wipe substrate.

The thickening system should be compatible with the compounds used in the present disclosure; that is, the thickening system, when used in combination with the skin microbiota balancing agent, should not precipitate out, form a coacervate, or prevent a user from perceiving the conditioning benefit (or other desired benefit) to be gained from the composition. The thickening system may include a thickener which can provide both the thickening effect desired from the thickening system and a conditioning effect to the user's skin.

Thickeners may include, cellulosics, gums, acrylates, starches and various polymers. Suitable examples include but are not limited to hydroxethyl cellulose, xanthan gum, guar gum, potato starch, and corn starch. In some embodiments, PEG-150 stearate, PEG-150 distearate, PEG-175 diisostearate, polyglyceryl-10 behenate/eicosadioate, disteareth-100 IPDI, polyacrylamidomethylpropane sulfonic acid, butylated PVP, and combinations thereof may be suitable.

While the viscosity of the compositions will typically depend on the thickener used and the other components of the compositions, the thickeners of the compositions suitably provide for a composition having a viscosity in the range of greater than 1 cP to about 30,000 cP or more. In another embodiment, the thickeners provide compositions having a viscosity of from about 100 cP to about 20,000 cP. In yet another embodiment, thickeners provide compositions having a viscosity of from about 200 cP to about 15,000 cP. In embodiments where the compositions are included in a wipe, the viscosity may range from about 1 cP to about 2000 cP. In some embodiments, it is preferable to have a viscosity of the composition be less than 500 cP.

When including a thickening system, the compositions of the present disclosure can include the thickening system in an amount of no more than about 20% (by total weight of the composition), or from about 0.01% (by total weight of the composition) to about 20% (by total weight of the composition). In another aspect the thickening system is present in the antimicrobial composition in an amount of from about 0.10% (by total weight of the composition) to about 10% (by total weight of the composition), or from about 0.25% (by total weight of the composition) to about 5% (by total weight of the composition), or from about 0.5% (by total weight of the composition) to about 2% (by total weight of the composition).

In one embodiment, the compositions may include hydrophobic and hydrophilic ingredients, such as a lotion or cream. Generally, these emulsions have a dispersed phase and a continuous phase, and are generally formed with the addition of a surfactant or a combination of surfactants with varying hydrophilic/lipophilic balances (HLB). Suitable emulsifiers include surfactants having HLB values from 0 to 20, or from 2 to 18. Suitable non-limiting examples include Ceteareth-20, Cetearyl Glucoside, Ceteth-10, Ceteth-2, Ceteth-20, Cocamide MEA, Glyceryl Laurate, Glyceryl Stearate, PEG-100 Stearate, Glyceryl Stearate, Glyceryl Stearate SE, Glycol Distearate, Glycol Stearate, Isosteareth-20, Laureth-23, Laureth-4, Lecithin, Methyl Glucose Sesquistearate, Oleth-10, Oleth-2, Oleth-20, PEG-100 Stearate, PEG-20 Almond Glycerides, PEG-20 Methyl Glucose Sesquistearate, PEG-25 Hydrogenated Castor Oil, PEG-30 Dipolyhydroxystearate, PEG-4 Dilaurate, PEG-40 Sorbitan Peroleate, PEG-60 Almond Glycerides, PEG-7 Olivate, PEG-7 Glyceryl Cocoate, PEG-8 Dioleate, PEG-8 Laurate, PEG-8 Oleate, PEG-80 Sorbitan Laurate, Polysorbate 20, Polysorbate 60, Polysorbate 80, Polysorbate 85, Propylene Glycol Isostearate, Sorbitan Isostearate, Sorbitan Laurate, Sorbitan Monostearate, Sorbitan Oleate, Sorbitan Sesquioleate, Sorbitan Stearate, Sorbitan Trioleate, Stearamide MEA, Steareth-100, Steareth-2, Steareth-20, Steareth-21. The compositions can further include surfactants or combinations of surfactants that create liquid crystalline networks or liposomal networks. Suitable non-limiting examples include OLIVEM 1000 (INCI: Cetearyl Olivate (and) Sorbitan Olivate (available from HallStar Company (Chicago, Ill.)); ARLACEL LC (INCI: Sorbitan Stearate (and) Sorbityl Laurate, commercially available from Croda (Edison, N.J.)); CRYSTALCAST MM (INCI: Beta Sitosterol (and) Sucrose Stearate (and) Sucrose Distearate (and) Cetyl Alcohol (and) Stearyl Alcohol, commercially available from MMP Inc. (South Plainfield, N.J.)); UNIOX CRISTAL (INCI: Cetearyl Alcohol (and) Polysorbate 60 (and) Cetearyl Glucoside, commercially available from Chemyunion (Sao Paulo, Brazil)). Other suitable emulsifiers include lecithin, hydrogenated lecithin, lysolecithin, phosphatidylcholine, phospholipids, and combinations thereof.

Gelling Agents

In some embodiments in which the composition is in the form of a gel, the disperse phase of the gel may be formed from any of a variety of different gelling agents, including temperature responsive (“thermogelling”) compounds, ion responsive compounds, and so forth. Thermogelling systems, for instance, respond to a change in temperature (e.g., increase in temperature) by changing from a liquid to a gel. Generally speaking, the temperature range of interest is from about 25° C. to about 40° C., in some embodiments from about 35° C. to about 39° C., and in one particular embodiment, at the human body temperature (about 37° C.). In some cases, thermogelling block copolymers, graft copolymers, and/or homopolymers may be employed. For example, polyoxyalkylene block copolymers may be used in some embodiments of the present invention to form a thermo-gelling composition. Suitable thermo-gelling compositions may include, for example, homopolymers, such as poly(N-methyl-N-n-propylacrylamide), poly(N-n-propylacrylamide), poly(N-methyl-N-isopropylacrylamide), poly(N-n-propylmethacrylamide), poly(N-isopropylacrylamide), poly(N,n-diethylacrylamide); poly(N-isopropylmethacrylamide), poly(N-cyclopropylacrylamide), poly(N-ethylmethyacrylamide), poly(N-methyl-N-ethylacrylamide), poly(N-cyclopropylmethacrylamide), and poly(N-ethylacrylamide). Still other examples of suitable thermogelling polymers may include cellulose ether derivatives, such as hydroxypropyl cellulose, methyl cellulose, hydroxypropylmethyl cellulose, and ethylhydroxyethyl cellulose. Moreover thermogelling polymers may be made by preparing copolymers between (among) monomers, or by combining such homopolymers with other water-soluble polymers, such as acrylic monomers (e.g., acrylic or methacrylic acid, acrylate or methacrylate, acrylamide or methacrylamide, and derivatives thereof).

Ion Responsive Compounds

The compositions of the present invention may also include an ion responsive compound. Such compounds are generally well known in the art, and tend to form a gel in the presence of certain ions or at a certain pH. For instance, one suitable class of ion responsive compounds that may be employed in the present invention is anionic polysaccharides. Anionic polysaccharides may form a three-dimensional polymer network that functions as the disperse phase of the gel. Generally speaking, anionic polysaccharides include polysaccharides having an overall anionic charge, as well as neutral polysaccharides that contain anionic functional groups.

Antimicrobial Agents

In some embodiments, the composition may include one or more antimicrobial agents to increase shelf life. Some suitable antimicrobial agents that may be used in the present disclosure include traditional antimicrobial agents. As used herein, “traditional antimicrobial agents” means compounds that have been historically recognized by regulatory bodies as providing an antimicrobial effect, such as those listed in the European Union's Annex V list of preservatives allowed in cosmetics products. Traditional antimicrobial agents include, but are not limited to: propionic acid and salts thereof; salicylic acid and salts thereof; sorbic acid and salts thereof; benzoic acid and salts and esters thereof; formaldehyde; paraformaldehyde; o-phenylphenol and salts thereof; zinc pyrithione; inorganic sulfites; hydrogen sulfites; chlorobutanol; benzoic parabens, such as methylparaben, propylparaben, butylparaben, ethylparaben, isopropylparaben, isobutylparaben, benzylparaben, sodium methylparaben and sodium propylparaben; dehydroacetic acid and salts thereof; formic acid and salts thereof; dibromohexamidine isethionate; thimerosal; phenylmercuric salts; undecylenic acid and salts thereof; hexetidine; 5-bromo-5-nitro-1,3-dioxane; 2-bromo-2-nitropropane-1,3,-diol; dichlorobenzyl alcohol; triclocarban; p-chloro-m-cresol; triclosan; chloroxylenol; imidazolidinyl urea; polyaminopropyl biguanide; phenoxyethanol, methenamine; quaternium-15; climbazole; DMDM hydantoin; benzyl alcohol; piroctone olamine; bromochlorophene; o-cymen-5-ol; methylchloroisothiazolinone; methylisothiazolinone; chlorophene; chloroacetamide; chlorhexidine; chlorhexidine diacetate; chlorhexidine digluconate; chlorhexidine dihydrochloride; phenoxyisopropanol; alkyl (C12-C22) trimethyl ammonium bromide and chlorides; dimethyl oxazolidine; diazolidinyl urea; hexamidine; hexamidine diisethionate; glutaral; 7-ethylbicyclooxazolidine; chlorphenesin; sodium hydroxymethylglycinate; silver chloride; benzethonium chloride; benzalkonium chloride; benzalkonium bromide; benzylhemiformal; iodopropynyl butylcarbamate; ethyl lauroyl arginate HCl; citric acid and silver citrate.

Other antimicrobial agents that may be added to the compositions of the present disclosure include non-traditional antimicrobial agents that are known to exhibit antimicrobial effects in addition to their primary functions, but that have not historically been recognized as antimicrobial agents by regulatory bodies (such as on the European Union's Annex V list). Examples of these non-traditional antimicrobial agents include, but are not limited to, hydroxyacetophenone, caprylyl glycol, sodium coco-PG dimonium chloride phosphate, phenylpropanol, lactic acid and salts thereof, caprylhydroxamic acid, levulinic acid and salts thereof, sodium lauroyl lactylate, phenethyl alcohol, sorbitan caprylate, glyceryl caprate, glyceryl caprylate, ethylhexylglycerin, p-anisic acid and salts thereof, gluconolactone, decylene glycol, 1,2-hexanediol, glucose oxidase and lactoperoxidase, leuconostoc/radish root ferment filtrate and glyceryl laurate.

The amount of the antimicrobial agents in the compositions is dependent on the relative amounts of other components present within the composition. For example, in some embodiments, an antimicrobial agent can be present in the compositions in an amount between about 0.001% to about 5% (by total weight of the composition), in some embodiments between about 0.01 to about 3% (by total weight of the composition), and in some embodiments, between about 0.05% to about 1.0% (by total weight of the composition). In some embodiments, the antimicrobial agent can be present in the composition in an amount less than 0.2% (by total weight of the composition). However, in some embodiments, the composition can be substantially free of any antimicrobial agents. Thus, in some embodiments, the composition does not include a traditional antimicrobial agent or a non-traditional antimicrobial agent.

Adjunct Ingredients

The compositions of the present disclosure may additionally include adjunct ingredients conventionally found in cosmetic, pharmaceutical, medical, household, industrial, or personal care compositions/products in an established fashion and at established levels. For example, the compositions may comprise additional compatible pharmaceutically active and compatible materials for combination therapy, such as antioxidants, anti-parasitic agents, antipruritics, antifungals, antiseptic actives, biological actives, astringents, keratolytic actives, local anaesthetics, anti-stinging agents, anti-reddening agents, skin soothing agents, external analgesics, film formers, skin exfoliating agents, sunscreens, and combinations thereof.

Other suitable additives that may be included in the compositions of the present disclosure include compatible colorants, deodorants, emulsifiers, anti-foaming agents (when foam is not desired), lubricants, skin conditioning agents, skin protectants and skin benefit agents (e.g., aloe vera and tocopheryl acetate), solvents (e.g., water soluble glycol and glycol ethers, glycerin, water soluble polyethylene glycols, water soluble polyethylene glycol ethers, water soluble polypropylene glycols, water soluble polypropylene glycol ethers, dimethylisosorbide), solubilizing agents, suspending agents, builders, (e.g., alkali and alkaline earth metal salts of carbonate, bicarbonate, phosphate, hydrogen phosphate, dihydrogen phosphate, sulfate hydrogen sulfate), wetting agents, pH adjusting ingredients (a suitable pH range of the compositions can be from about 3.5 to about 8), chelators, propellants, dyes and/or pigments, and combinations thereof.

Another component that may be suitable for addition to the compositions is a fragrance. Any compatible fragrance may be used. Typically, the fragrance is present in an amount from about 0% (by weight of the composition) to about 5% (by weight of the composition), and more typically from about 0.01% (by weight of the composition) to about 3% (by weight of the composition). In one desirable embodiment, the fragrance will have a clean, fresh and/or neutral scent to create an appealing delivery vehicle for the end consumer.

Organic sunscreens that may be present in the compositions include ethylhexyl methoxycinnamate, avobenzone, octocrylene, benzophenone-4, phenylbenzimidazole sulfonic acid, homosalate, oxybenzone, benzophenone-3, ethylhexyl salicylate, and mixtures thereof.

As previously noted herein, the compositions of the present disclosure may be applied to a delivery mechanism such as a substrate, which in-turn may be used to deliver and/or apply the prebiotic composition to a user's skin. Suitable substrates include a web, such as a wet laid tissue web or air laid web, gauze, cotton swab, transdermal patch, container or holder. Various webs that the composition can be applied to can provide products in the form of wipes, facial tissue, bath tissue, paper towels, napkins, diapers, diaper pants, feminine hygiene products (tampons, pads), gloves, socks, masks or combinations thereof. In some embodiments, a substrate that the composition is applied to can form part of an absorbent article. For example, the composition could be applied to a substrate that can form at least a portion of a bodyside liner of an absorbent article. Particularly preferred applicators include fibrous webs, including flushable and non-flushable cellulosic webs and nonwoven webs of synthetic fibrous material. Useful webs may be wet laid, air laid, meltblown, or spunbonded. Suitable synthetic fibrous material includes meltblown polyethylene, polypropylene, copolymers of polyethylene and polypropylene, bicomponent fibers including polyethylene or polypropylene, and the like. Useful nonwoven webs may be meltblown, coform, spunbond, airlaid, hydroentangled nonwovens, spunlace, bonded carded webs.

In certain embodiments, particularly those in which the composition is applied to a web, it may be desirable that the formulation provide certain physical attributes, such as having a smooth, lubricious, non-greasy feel; the ability to at least partially transfer from the web to the user's skin; the capability to be retained on the web at about room temperature; or the ability to be compatible with the web manufacturing process. In certain embodiments, it is preferred that at least a portion of the composition is transferred from the tissue to the user's skin in use.

The composition may be applied to a web during formation of the web or after the web has been formed and dried, often referred to as off-line or post-treatment. Suitable methods of applying the composition to a web include methods known in the art such as gravure printing, flexographic printing, spraying, WEKO™, slot die coating, or electrostatic spraying. One particularly preferred method of off-line application is rotogravure printing.

In those instances where the composition is added to the web during formation of the web and prior to drying, it may be preferred to employ an application method that incorporates the composition on the surface of the web. One method of adding the prebiotic to the web surface is by applying the composition during creping of the tissue web. Surprisingly, the composition itself may be used as a creping composition or may be combined with other well-known creping compositions to apply the composition to a tissue web without significantly degrading important web properties such as strength, stiffness or sloughing.

In some embodiments where the composition is applied to a delivery mechanism such as a substrate, the composition can be applied in an add-on amount ranging from about 1% to about 500%, or from about 30% to about 400%, or from about 100% to about 350%. Of course, it is contemplated that the composition may be applied to a delivery substance outside of this range and still be within the scope of this disclosure.

Fibrous webs comprising a composition made according to the present disclosure can be incorporated into multi-ply products. For instance, in one aspect, a fibrous web made according to the present disclosure can be attached to one or more other fibrous webs to form a wiping product having desired characteristics. The other webs laminated to the fibrous web of the present disclosure can be, for instance, a wet-creped web, a calendered web, an embossed web, a through-air dried web, a creped through-air dried web, an uncreped through-air dried web, an airlaid web, and the like, and may or may not comprise any skin microbiota balancing agents.

Processes for producing airlaid non-woven basesheets are described in, for example, published U.S. Pat. App. No. 2006/0008621, herein incorporated by reference to the extent it is consistent herewith.

Test Methods

Carbohydrate Screening Test Method

The study employed 10 representative skin bacteria; Staphylococcus epidermidis ATCC 14990, Staphylococcus epidermidis M23864:W2, Staphylococcus aureus ATCC 6538, Staphylococcus aureus ATCC 25904 (Newman), Pseudomonas aeruginosa ATCC 9027, Micrococcus luteus ATCC 48732, Micrococcus luteus SK58, Klebsiella pneumoniae ATCC BAA-2146, Corynebacterium amycolatum SK46, and Corynebacterium minutissimum ATCC 23348. Cultures were started from freezer bead stocks by placing a single bead into 20 mL of tryptic soy broth (TSB) for all organisms except C. amycolatum SK46 and C. minutissimum ATCC 23348, which were placed into brain heart infusion (BHI) broth. Cultures were grown for about 24 hr at 37° C. except M. luteus ATCC 49732 and M. luteus SK58, which were grown at 30° C. A passage of the culture was made using a sterile swab and streaked onto two M9*2 agar plates to prepare the incubated culture for the phenotype microarray pate according to Table 4. Plates were incubated for about 24 hr at 37° C. except for M. luteus ATCC 49732 and M. luteus SK58, which were incubated at 30° C. for 24 hr.

TABLE 4
Preparation of the incubated culture for the phenotype microarray pate

| | M9*1 Media | | M9*2 Media | | |
|---|---|---|---|---|---|
| Ingredient | Amount | Unit | Amount | Unit | Alternative |
| Bacto Agar | — | — | 15 | G | — |
| Casamino | — | — | 10 | G | — |
| Acids | | | | | |
| Na₂HPO₄7H₂O | 12.8 | G | 12.8 | G | 200 mL of 5X |
| KH₂PO₄ | 3 | G | 3 | G | M9 Salts |
| NaCl | 0.5 | G | 0.5 | G | solution |
| NH₄Cl | 1 | G | 1 | G | |
| Glucose | — | — | 200 | G | 20 mL of 20% |
| | | | | | Glucose |
| | | | | | Solution |
| MGSO₄ | 0.24 | G | 0.24 | G | 2 mL 1M of |
| | | | | | MGSO₄ solution |
| CaCl₂ | 0.0011 | G | 0.0011 | G | 0.1 mL 1M |
| | | | | | CaCl₂ solution |

| | |
|---|---|
| Sterile DI Water | QS to 1 L |

Phenotype Microarray Set Up

All cultures were harvested and washed before inoculation into the carbon microarray plate (BiOLOG, Hayward Calif.), with Biolog Plates PM1 and PM2 each having a source definition of Carbon. The bacteria on the M9*2 agar was suspended via agitation with an inoculating loop in 1 mL of M9*1 media for collection in a 1.7 mL micro centrifuge tube, as noted in Table 4. The bacteria was suspended again in M9*1 media, agitated, and collected into the same 1.7 mL microcentrifuge tube used in the previous step. The tube was centrifuged for 5 minutes at 4,000×G, supernatant decanted, the pellet was resuspended in 1 mL of M9*1 media, and vortex. This washing process was repeated for a total of 4 times.

A tenfold dilution in M9*1 media was performed using 1 mL of the washed bacterial culture. Of the tenfold dilution 200 μL was placed in an untreated 96-well plate (Falcon, Pasadena Tx.) for an initial optical density (OD) reading at a wavelength of 600 nm. The OD was used to correlate to colony forming units (CFU) per milliliter by using linear regression lines for each organism. Refer to Appendix B for the equations used for each organism. Utilizing the output from the linear regression lines all organisms were diluted such that 10⁴ CFU/mL was reached.

The inoculating media was supplemented with 0.75%(w/v) BHI for S. epidermidis M23864:W2, S. epidermidis ATCC 14990, and C. minutissimum ATCC 23348. For S. aureus ATCC 6538 and S. aureus ATCC 25904 the inoculating media was supplemented with 0.25% (w/v) BHI (Table 5). M9*1 media was used as the inoculating media for P. aeruginosa ATCC 9027, M. luteus ATCC 49732, M. luteus SK58, and K. pneumoniae BAA-2146. A multichannel pipette was used to place 100 μL of inoculum into each well targeting approximately 10′ CFU/well in the phenotype microarray plate. The plate cover was treated with anti-fog (Xodus Medical Inc., New Kensington, Pa.) to help reduce condensation. The phenotype microarray plate was placed into a Gemini 3M plate reader (Molecular Devices, Sunnyvale, Calif.) with SoftMax Pro 6.3 software (Molecular Devices, Sunnyvale, Calif.). The following settings were used, a kinetic read every 20 min for 36 hr, a wavelength of 600 nm, and a temperature of 33° C.

TABLE 5
Inoculating media for S. epidermis M23864:W2,
S. epidermis ATCC 14990, C. minutissimum ATCC 23348,
S. aureus ATCC 6538, and S. aureus ATCC 25904

| Plate Type | Ingredient | Amount | Unit |
|---|---|---|---|
| Carbon | Sterile H₂O | 760.34 | mL |
| | Casamino Acids | 10 | G |
| | M9 Salts 5x Solution | 200 | mL |
| | 20% Glucose | 20 | mL |
| | 1M MgSO₄ solution | 2 | mL |
| | 1M CaCl₂ solution | 0.1 | mL |

Controls for Phenotype Microarray

An untreated 96-well plate was used as the control. All wells were loaded with 200 μL of media specified in Table 6. The control plate contained a media blank for all media used in the control plate and that same media inoculated with bacteria targeting approximately 10′ CFU/well in the plate. M9*1 was used as the negative growth control. TSB was used as the positive growth control with the exception of C. minutissimum ATCC23348 and C. amycolatum SK46 where BHI was used. The microarray plate inoculating media (missing carbon) was used as a background control. Growth should not be observed for this media. The final control was the microarray plate inoculating media containing carbon. Growth should be observed for this media. The control plate was incubated at 37° C. for 36 hr except M. luteus ATCC 49732 and M. luteus SK58. They were incubated at 33° C. for 36 hr. Following incubation an endpoint absorbance read was taken at OD 600 nm. If multiple organisms shared the same inoculating media one plate blank was sufficient for those organisms.

TABLE 6
Control Plate Layout

| | 1 | 2 | 3 | 4 | 5 | 6 | 7 | 8 | 9 | 10 | 11 | 12 |
|---|---|---|---|---|---|---|---|---|---|---|---|---|
| A | Inoculating | Full | M9*1 | TSB/BHI | | | | | | | | |
| B | media | strength | media | media | | | | | | | | |
| C | blank | media | blank | blank | | | | | | | | |
| D | | blank | | | | | | | | | | |
| E | Inoculating | Full | M9*1 | TSB/BHI | | | | | | | | |
| F | media with | strength | media | media | | | | | | | | |
| G | bacteria | media | with | with | | | | | | | | |
| H | | with | bacteria | bacteria | | | | | | | | |
| | | bacteria | | | | | | | | | | |

Spread plating was utilized to confirm the inoculum bacterial concentration. The dilutions were plated onto TSA, in duplicate, with the exception of C. minutissimum ATCC23348 and C. amycolatum SK46, which were plated onto BHI agar. All plates were incubated at 37° C. for 36 hr, except M. luteus ATCC 49732 and M. luteus SK58. They were incubated at 33° C. for 36 hr.

Analysis of Control Plate

The average OD of the media blanks (wells A1-D1, A2-D2, A3-D3, and A4-D4) were expected to have low OD values. The average OD of wells E1-H1 was expected to have a value similar to that of A1-D1 (inoculum lacking carbon). The average OD of E2-H2 (inoculum containing carbon) was expected to have a high value as compared to A2-D2. The average OD of E3-H3 (media lacking carbon & nitrogen) was expected to have a similar value as the average OD of A3-D3. Lastly, the average OD of E4-H4 (positive growth control) was expected to have the highest value.

Analysis of Phenotype Microarray Plate

Biochemical utilization patterns were analyzed using SoftMax Pro 6.3 software. Background was eliminated by subtracting the negative control (well A1) from all other wells. To do so in SoftMax, “Template Editor” was chosen, the Al cell was clicked, “Plate Blank” was selected, and “okay” was hit. All curves were normalized for better comparison by clicking “Reduction,” number 2 “set first data point to zero” was checked, and “okay” was clicked. The wells were highlighted seven at a time and while highlighted double clicked on. All seven curves were displayed on the screen. Maximum OD's were observed by hovering over the highest part of the curve with the mouse, the second number displayed is the OD. The graphs that reached less than 0.05 OD units above zero, decreased below zero, or did not show traditional growth curve trends were considered negative for supporting growth. Graphs that showed traditional growth curve trends and reached more than 0.05 OD units above zero were considered positive and supported growth. The positive hits were sorted in no particular way.

### Embodiments

In view of the foregoing description and examples, the present disclosure provides the following embodiments.

Embodiment 1: A composition for providing or maintaining a healthy skin microbiota, the composition comprising: a carrier; and a skin microbiota balancing agent, the skin microbiota balancing agent including at least one combination of carbohydrate sources including a first carbohydrate source and a second carbohydrate source, wherein the skin microbiota balancing agent is configured to provide at least two of the following desired ratios: a first desired ratio of Corynebacterium to Staphylococcus, a second desired ratio of Corynebacterium to Micrococcus, and a third desired ratio of Staphylococcus to Micrococcus.

Embodiment 2: The composition of embodiment 1, wherein the first desired ratio of Corynebacterium to Staphylococcus is 1.3, the second desired ratio of Corynebacterium to Micrococcus is 1.4, and the third desired ratio of Staphylococcus to Micrococcus is 1.1.

Embodiment 3: The composition of embodiment 1 or 2, wherein the at least one combination of carbohydrate sources is selected from the group consisting of: D-Alanine+γ-Cyclodextrin, D-Alanine+Sebacic Acid, D-Alanine+2,3-Butanediol, D-Aspartic Acid+Oxalic Acid, D-Aspartic Acid+Pectin, D-Threonine+3-Methyl Glucose, D-Threonine+Pectin, Fumaric Acid+α-Cyclodextrin, Fumaric Acid+Mannan, L-Alanyl-Glycine+3-Methyl Glucose, L-Alanyl-Glycine+Oxalic Acid, L-Alanyl-Glycine+Pectin, L-Malic Acid+Mannan, D-Saccharic Acid+Pectin+L-Alanine, Succinic Acid+3-Methyl Glucose+D-Glucosaminic Acid, Succinic Acid+3-Methyl Glucose+L-Alanine, Succinic Acid+3-Methyl Glucose+Glycyl-L-Aspartic Acid, Succinic Acid+Pectin+D-Glucosaminic Acid, Succinic Acid+Pectin+Glycyl-L-Aspartic Acid, Tween 40+3-Methyl Glucose+D-glucosaminic Acid, Tween 40+3-Methyl Glucose+L-Alanine, Tween 40+3-Methyl Glucose+Glycyl-L-Aspartic Acid, Tween 40+Pectin+D-glucosaminic Acid, Tween 40+Pectin+Glycyl-L-Aspartic Acid, α-Keto-Glutaric Acid+γ-Cyclodextrin+Glycyl-L-Aspartic Acid, α-Keto-Glutaric Acid+Inulin+D-glucosaminic Acid, α-Keto-Glutaric Acid+3-Methyl Glucose+Glycyl-L-Aspartic Acid, α-Keto-Glutaric Acid+γ-Amino Butyric Acid+Glycyl-L-Aspartic Acid, α-Keto-Glutaric Acid+Sebacic Acid+D-glucosaminic Acid, α-Keto-Glutaric Acid+2,3-Butanediol+Glycyl-L-Aspartic Acid, α-Keto-Glutaric Acid+3-Hydroxy-2-Butanone+D-glucosaminic Acid, α-Keto-Butyric Acid+α-Cyclodextrin+N-Acetyl-D-Galactosamine, α-Keto-Butyric Acid+Inulin+D-glucosaminic Acid, α-Keto-Butyric Acid+Inulin+Glycyl-L-Aspartic Acid, α-Keto-Butyric Acid+3-Methyl Glucose+D-glucosaminic Acid, α-Keto-Butyric Acid+3-Methyl Glucose+Glycyl-L-Aspartic Acid, α-Keto-Butyric Acid+Oxalic Acid+L-Alanine, α-Keto-Butyric Acid+Sebacic Acid+D-glucosaminic Acid, α-Keto-Butyric Acid+Sebacic Acid+Glycyl-L-Aspartic Acid, α-Keto-Butyric Acid+L-Homoserine+N-Acetyl-D-Galactosamine, α-Keto-Butyric Acid+2,3-Butanediol+N-Acetyl-D-Galactosamine, α-Keto-Butyric Acid+3-Hydroxy-2-Butanone+N-Acetyl-D-Galactosamine, m-Tartaric Acid+Pectin+D-Glucosaminic Acid, m-Tartaric Acid+Pectin+Glycyl-L-Aspartic Acid, α-Hydroxy Butyric Acid+Inulin+D-glucosaminic Acid, α-Hydroxy Butyric Acid+Inulin+Glycyl-L-Aspartic Acid, α-Hydroxy Butyric Acid+3-Methyl Glucose+D-glucosaminic Acid, α-Hydroxy Butyric Acid+3-Methyl Glucose+Glycyl-L-Aspartic Acid, α-Hydroxy Butyric Acid+Oxalic Acid+L-Alanine, α-Hydroxy Butyric Acid+Sebacic Acid+D-glucosaminic Acid, α-Hydroxy Butyric Acid+Sebacic Acid+Glycyl-L-Aspartic Acid, α-Hydroxy Butyric Acid+2,3-Butanediol+N-Acetyl-D-Galactosamine, α-Hydroxy Butyric Acid+3-Hydroxy-2-Butanone+N-Acetyl-D-Galactosamine, Citric Acid+α-Cyclodextrin+Glycyl-L-Glutamic Acid, Citric Acid+α-Cyclodextrin+Tricarballylic Acid, Citric Acid+Mannan+Glycyl-L-Glutamic Acid, Citric Acid+Mannan+Tricarballylic Acid, Citric Acid+Oxalic Acid+L-Alanine, Citric Acid+Pectin+L-Alanine, Bromo Succinic Acid+2,3-Butanediol+N-Acetyl-D-Galactosamine, Bromo Succinic Acid+2,3-Butanediol+glycyl-L-Aspartic Acid, Bromo Succinic Acid+3-Hydroxy-2-Butanone+N-Acetyl-D-Galactosamine, Bromo Succinic Acid+3-Hydroxy-2-Butanone+Glycyl-L-Aspartic Acid, Bromo Succinic Acid+Pectin+L-Alanine, Mucic Acid+Inulin+D-glucosaminic Acid, Mucic Acid+Inulin+Glycyl-L-Aspartic Acid, Mucic Acid+3-Methyl Glucose+D-glucosaminic Acid, Mucic Acid+3-Methyl Glucose+Glycyl-L-Aspartic Acid, Mucic Acid+Oxalic Acid+L-Alanine, Mucic Acid+Sebacic Acid+D-glucosaminic Acid, Mucic Acid+Sebacic Acid+Glycyl-L-Aspartic Acid, Mucic Acid+2,3-Butanediol+N-Acetyl-D-Galactosamine, Mucic Acid+3-Hydroxy-2-Butanone+N-Acetyl-D-Galactosamine, Glyocylic Acid+Inulin+Glycyl-L-Aspartic Acid, Glyocylic Acid+3-Methyl Glucose+D-glucosaminic Acid, Glyocylic Acid+3-Methyl Glucose+Glycyl-L-Aspartic Acid, Glyocylic Acid+Oxalic Acid+L-Alanine, Glyocylic Acid+Sebacic Acid+D-glucosaminic Acid, Glyocylic Acid+Sebacic Acid+Glycyl-L-Aspartic Acid, Glyocylic Acid+2,3-Butanediol+N-Acetyl-D-Galactosamine, Glyocylic Acid+3-Hydroxy-2-Butanone+N-Acetyl-D-Galactosamine, Phenylethyl-amine+3-Methyl Glucose+D-glucosaminic Acid, Phenylethyl-amine+3-Methyl Glucose+N-Acetyl-D-Galactosamine, Phenylethyl-amine+3-Methyl Glucose+Glycyl-L-Aspartic Acid, Phenylethyl-amine+Oxalic Acid+L-Alanine, Phenylethyl-amine+Oxalic Acid+N-Acetyl-D-Galactosamine, Phenylethyl-amine+Pectin+N-Acetyl-D-Galactosamine, D-Alanine+3-Methyl Glucose, D-Alanine+Oxalic Acid, L-Alanyl-Glycine+γ-Cyclodextrin, L-Alanyl-Glycine+Inulin, L-Alanyl-Glycine+Sebacic Acid, L-Alanyl-Glycine+2,3-Butanediol, L-Alanyl-Glycine+3-Hydroxy 2-Butanone, D-Saccharic Acid+Oxalic Acid+L-Alanine, Succinic Acid+Oxalic Acid+L-Alanine, Succinic Acid+Pectin+L-Alanine, Tween 40+Oxalic Acid+L-Alanine, Tween 40+Pectin+L-Alanine, α-Keto-Glutaric Acid+Inulin+Glycyl-L-Aspartic Acid, α-Keto-Glutaric Acid+Sebacic Acid+Glycyl-L-Aspartic Acid, α-Keto-Glutaric Acid+3-Hydroxy-2-Butanone+Glycyl-L-Aspartic Acid, m-Tartaric Acid+Oxalic Acid+L-Alanine, m-Tartaric Acid+Pectin+L-Alanine, Bromo Succinic Acid+γ-Cyclodextrin+D-glucosaminic Acid, Bromo Succinic Acid+γ-Cyclodextrin+Glycyl-L-Aspartic Acid, Bromo Succinic Acid+Inulin+D-glucosaminic Acid, Bromo Succinic Acid+Inulin+Glycyl-L-Aspartic Acid, Bromo Succinic Acid+3-Methyl Glucose+D-glucosaminic Acid, Bromo Succinic Acid+3-Methyl Glucose+Glycyl-L-Aspartic Acid, Bromo Succinic Acid+Sebacic Acid+D-glucosaminic Acid, Bromo Succinic Acid+Sebacic Acid+glycyl-L-Aspartic Acid, Bromo Succinic Acid+2,3-Butanediol+D-glucosaminic Acid, Bromo Succinic Acid+3-Hydroxy-2-Butanone+D-glucosaminic Acid, Bromo Succinic Acid+Pectin+D-Glucosaminic Acid, Bromo Succinic Acid+Pectin+Glycyl-L-Aspartic Acid, Phenylethyl-amine+α-Cyclodextrin+N-Acetyl-D-Galactosamine, Phenylethyl-amine+Turanose+N-Acetyl-D-Galactosamine, Phenylethyl-amine+L-Homoserine+N-Acetyl-D-Galactosamine, Phenylethyl-amine+2,3-Butanediol+N-Acetyl-D-Galactosamine, and Phenylethyl-amine+3-Hydroxy-2-Butanone+N-Acetyl-D-Galactosamine.

Embodiment 4: The composition of embodiment 1 or 2, wherein the skin microbiota balancing agent is configured to provide the first desired ratio of Corynebacterium to Staphylococcus, the second desired ratio of Corynebacterium to Micrococcus, and the third desired ratio of Staphylococcus to Micrococcus.

Embodiment 5: The composition of embodiment 4, wherein the at least one combination of carbohydrate sources is selected from the group consisting of: D-Alanine+3-Methyl Glucose, D-Alanine+Oxalic Acid, L-Alanyl-Glycine+γ-Cyclodextrin, L-Alanyl-Glycine+Inulin, L-Alanyl-Glycine+Sebacic Acid, L-Alanyl-Glycine+2,3-Butanediol, L-Alanyl-Glycine+3-Hydroxy 2-Butanone, D-Saccharic Acid+Oxalic Acid+L-Alanine, Succinic Acid+Oxalic Acid+L-Alanine, Succinic Acid+Pectin+L-Alanine, Tween 40+Oxalic Acid+L-Alanine, Tween 40+Pectin+L-Alanine, α-Keto-Glutaric Acid+Inulin+Glycyl-L-Aspartic Acid, α-Keto-Glutaric Acid+Sebacic Acid+Glycyl-L-Aspartic Acid, α-Keto-Glutaric Acid+3-Hydroxy-2-Butanone+Glycyl-L-Aspartic Acid, m-Tartaric Acid+Oxalic Acid+L-Alanine, m-Tartaric Acid+Pectin+L-Alanine, Bromo Succinic Acid+γ-Cyclodextrin+D-glucosaminic Acid, Bromo Succinic Acid+γ-Cyclodextrin+Glycyl-L-Aspartic Acid, Bromo Succinic Acid+Inulin+D-glucosaminic Acid, Bromo Succinic Acid+Inulin+Glycyl-L-Aspartic Acid, Bromo Succinic Acid+3-Methyl Glucose+D-glucosaminic Acid, Bromo Succinic Acid+3-Methyl Glucose+Glycyl-L-Aspartic Acid, Bromo Succinic Acid+Sebacic Acid+D-glucosaminic Acid, Bromo Succinic Acid+Sebacic Acid+glycyl-L-Aspartic Acid, Bromo Succinic Acid+2,3-Butanediol+D-glucosaminic Acid, Bromo Succinic Acid+3-Hydroxy-2-Butanone+D-glucosaminic Acid, Bromo Succinic Acid+Pectin+D-Glucosaminic Acid, Bromo Succinic Acid+Pectin+Glycyl-L-Aspartic Acid, Phenylethyl-amine+α-Cyclodextrin+N-Acetyl-D-Galactosamine, Phenylethyl-amine+Turanose+N-Acetyl-D-Galactosamine, Phenylethyl-amine+L-Homoserine+N-Acetyl-D-Galactosamine, Phenylethyl-amine+2,3-Butanediol+N-Acetyl-D-Galactosamine, and Phenylethyl-amine+3-Hydroxy-2-Butanone+N-Acetyl-D-Galactosamine.

Embodiment 6: The composition of any one of the preceding embodiments, wherein the composition is applied to a component of an absorbent article or to a wipe.

Embodiment 7: The composition of any one of the preceding embodiments, wherein the composition is in the form of a liquid or cream.

Embodiment 8: A composition for providing or maintaining a healthy skin microbiota, the composition comprising: a carrier; and a skin microbiota balancing agent, the skin microbiota balancing agent including at least one combination of carbohydrate sources, the combination of carbohydrate sources being selected from the group consisting of: D-Alanine+3-Methyl Glucose, D-Alanine+Oxalic Acid, L-Alanyl-Glycine+γ-Cyclodextrin, L-Alanyl-Glycine+Inulin, L-Alanyl-Glycine+Sebacic Acid, L-Alanyl-Glycine+2,3-Butanediol, L-Alanyl-Glycine+3-Hydroxy 2-Butanone, D-Saccharic Acid+Oxalic Acid+L-Alanine, Succinic Acid+Oxalic Acid+L-Alanine, Succinic Acid+Pectin+L-Alanine, Tween 40+Oxalic Acid+L-Alanine, Tween 40+Pectin+L-Alanine, α-Keto-Glutaric Acid+Inulin+Glycyl-L-Aspartic Acid, α-Keto-Glutaric Acid+Sebacic Acid+Glycyl-L-Aspartic Acid, α-Keto-Glutaric Acid+3-Hydroxy-2-Butanone+Glycyl-L-Aspartic Acid, m-Tartaric Acid+Oxalic Acid+L-Alanine, m-Tartaric Acid+Pectin+L-Alanine, Bromo Succinic Acid+γ-Cyclodextrin+D-glucosaminic Acid, Bromo Succinic Acid+γ-Cyclodextrin+Glycyl-L-Aspartic Acid, Bromo Succinic Acid+Inulin+D-glucosaminic Acid, Bromo Succinic Acid+Inulin+Glycyl-L-Aspartic Acid, Bromo Succinic Acid+3-Methyl Glucose+D-glucosaminic Acid, Bromo Succinic Acid+3-Methyl Glucose+Glycyl-L-Aspartic Acid, Bromo Succinic Acid+Sebacic Acid+D-glucosaminic Acid, Bromo Succinic Acid+Sebacic Acid+glycyl-L-Aspartic Acid, Bromo Succinic Acid+2,3-Butanediol+D-glucosaminic Acid, Bromo Succinic Acid+3-Hydroxy-2-Butanone+D-glucosaminic Acid, Bromo Succinic Acid+Pectin+D-Glucosaminic Acid, Bromo Succinic Acid+Pectin+Glycyl-L-Aspartic Acid, Phenylethyl-amine+a-Cyclodextrin+N-Acetyl-D-Galactosamine, Phenylethyl-amine+Turanose+N-Acetyl-D-Galactosamine, Phenylethyl-amine+L-Homoserine+N-Acetyl-D-Galactosamine, Phenylethyl-amine+2,3-Butanediol+N-Acetyl-D-Galactosamine, and Phenylethyl-amine+3-Hydroxy-2-Butanone+N-Acetyl-D-Galactosamine.

Embodiment 9: The composition of embodiment 8, the skin microbiota balancing agent is configured to provide a first desired ratio of Corynebacterium to Staphylococcus, a second desired ratio of Corynebacterium to Micrococcus, and a third desired ratio of Staphylococcus to Micrococcus.

Embodiment 10: The composition of embodiment 9, wherein the first desired ratio of Corynebacterium to Staphylococcus is 1.3, the second desired ratio of Corynebacterium to Micrococcus is 1.4, and the third desired ratio of Staphylococcus to Micrococcus is 1.1.

Embodiment 11: The composition of any one of embodiments 8-10, wherein the wherein the composition is applied to a component of an absorbent article or to a wipe.

Embodiment 12: The composition of any one of embodiments 8-11, wherein the composition is in the form of a liquid or cream.

Embodiment 13: A method to provide or maintain a healthy skin microbiota on a subject, the method comprising: creating a prebiotic composition configured to maintain at least one of a first desired ratio of Corynebacterium to Staphylococcus, a second desired ratio of Corynebacterium to Micrococcus, and a third desired ratio of Staphylococcus to Micrococcus, the prebiotic composition comprising: a carrier; and a skin microbiota balancing agent comprising at least a first carbohydrate source, selecting the first carbohydrate source to maintain the at least one of the first desired ratio of Corynebacterium to Staphylococcus, the second desired ratio of Corynebacterium to Micrococcus, and the third desired ratio of Staphylococcus to Micrococcus; and providing instruction to apply the prebiotic composition to the subject.

Embodiment 14: The method of embodiment 13, further comprising: administering the prebiotic composition to the subject to provide or maintain a healthy skin microbiota in the subject.

Embodiment 15: The method of embodiment 13 or 14, wherein the first desired ratio of Corynebacterium to Staphylococcus is 1.3, the second desired ratio of Corynebacterium to Micrococcus is 1.4, and the third desired ratio of Staphylococcus to Micrococcus is 1.1.

Embodiment 16: The method of any one of embodiments 13-15, wherein the prebiotic composition is configured to maintain at least two of the first desired ratio of Corynebacterium to Staphylococcus, the second desired ratio of Corynebacterium to Micrococcus, and the third desired ratio of Staphylococcus to Micrococcus.

Embodiment 17: The method of any one of embodiments 13-16, wherein the skin microbiota balancing agent includes at least one combination of carbohydrate sources, the at least one combination of carbohydrate sources including the first carbohydrate source and a second carbohydrate source.

Embodiment 18: The method of embodiment 17, wherein the at least one combination of carbohydrate sources is selected from the group consisting of: D-Alanine+γ-Cyclodextrin, D-Alanine+Sebacic Acid, D-Alanine+2,3-Butanediol, D-Aspartic Acid+Oxalic Acid, D-Aspartic Acid+Pectin, D-Threonine+3-Methyl Glucose, D-Threonine+Pectin, Fumaric Acid+α-Cyclodextrin, Fumaric Acid+Mannan, L-Alanyl-Glycine+3-Methyl Glucose, L-Alanyl-Glycine+Oxalic Acid, L-Alanyl-Glycine+Pectin, L-Malic Acid+Mannan, D-Saccharic Acid+Pectin+L-Alanine, Succinic Acid+3-Methyl Glucose+D-Glucosaminic Acid, Succinic Acid+3-Methyl Glucose+L-Alanine, Succinic Acid+3-Methyl Glucose+Glycyl-L-Aspartic Acid, Succinic Acid+Pectin+D-Glucosaminic Acid, Succinic Acid+Pectin+Glycyl-L-Aspartic Acid, Tween 40+3-Methyl Glucose+D-glucosaminic Acid, Tween 40+3-Methyl Glucose+L-Alanine, Tween 40+3-Methyl Glucose+Glycyl-L-Aspartic Acid, Tween 40+Pectin+D-glucosaminic Acid, Tween 40+Pectin+Glycyl-L-Aspartic Acid, α-Keto-Glutaric Acid+y-Cyclodextrin+Glycyl-L-Aspartic Acid, α-Keto-Glutaric Acid+Inulin+D-glucosaminic Acid, α-Keto-Glutaric Acid+3-Methyl Glucose+Glycyl-L-Aspartic Acid, α-Keto-Glutaric Acid+γ-Amino Butyric Acid+Glycyl-L-Aspartic Acid, α-Keto-Glutaric Acid+Sebacic Acid+D-glucosaminic Acid, α-Keto-Glutaric Acid+2,3-Butanediol+Glycyl-L-Aspartic Acid, α-Keto-Glutaric Acid+3-Hydroxy-2-Butanone+D-glucosaminic Acid, α-Keto-Butyric Acid+α-Cyclodextrin+N-Acetyl-D-Galactosamine, α-Keto-Butyric Acid+Inulin+D-glucosaminic Acid, α-Keto-Butyric Acid+Inulin+Glycyl-L-Aspartic Acid, α-Keto-Butyric Acid+3-Methyl Glucose+D-glucosaminic Acid, α-Keto-Butyric Acid+3-Methyl Glucose+Glycyl-L-Aspartic Acid, α-Keto-Butyric Acid+Oxalic Acid+L-Alanine, α-Keto-Butyric Acid+Sebacic Acid+D-glucosaminic Acid, α-Keto-Butyric Acid+Sebacic Acid+Glycyl-L-Aspartic Acid, α-Keto-Butyric Acid+L-Homoserine+N-Acetyl-D-Galactosamine, α-Keto-Butyric Acid+2,3-Butanediol+N-Acetyl-D-Galactosamine, α-Keto-Butyric Acid+3-Hydroxy-2-Butanone+N-Acetyl-D-Galactosamine, m-Tartaric Acid+Pectin+D-Glucosaminic Acid, m-Tartaric Acid+Pectin+Glycyl-L-Aspartic Acid, α-Hydroxy Butyric Acid+Inulin+D-glucosaminic Acid, α-Hydroxy Butyric Acid+Inulin+Glycyl-L-Aspartic Acid, α-Hydroxy Butyric Acid+3-Methyl Glucose+D-glucosaminic Acid, α-Hydroxy Butyric Acid+3-Methyl Glucose+Glycyl-L-Aspartic Acid, α-Hydroxy Butyric Acid+Oxalic Acid+L-Alanine, α-Hydroxy Butyric Acid+Sebacic Acid+D-glucosaminic Acid, α-Hydroxy Butyric Acid+Sebacic Acid+Glycyl-L-Aspartic Acid, α-Hydroxy Butyric Acid+2,3-Butanediol+N-Acetyl-D-Galactosamine, α-Hydroxy Butyric Acid+3-Hydroxy-2-Butanone+N-Acetyl-D-Galactosamine, Citric Acid+α-Cyclodextrin+Glycyl-L-Glutamic Acid, Citric Acid+α-Cyclodextrin+Tricarballylic Acid, Citric Acid+Mannan+Glycyl-L-Glutamic Acid, Citric Acid+Mannan+Tricarballylic Acid, Citric Acid+Oxalic Acid+L-Alanine, Citric Acid+Pectin+L-Alanine, Bromo Succinic Acid+2,3-Butanediol+N-Acetyl-D-Galactosamine, Bromo Succinic Acid+2,3-Butanediol+glycyl-L-Aspartic Acid, Bromo Succinic Acid+3-Hydroxy-2-Butanone+N-Acetyl-D-Galactosamine, Bromo Succinic Acid+3-Hydroxy-2-Butanone+Glycyl-L-Aspartic Acid, Bromo Succinic Acid+Pectin+L-Alanine, Mucic Acid+Inulin+D-glucosaminic Acid, Mucic Acid+Inulin+Glycyl-L-Aspartic Acid, Mucic Acid+3-Methyl Glucose+D-glucosaminic Acid, Mucic Acid+3-Methyl Glucose+Glycyl-L-Aspartic Acid, Mucic Acid+Oxalic Acid+L-Alanine, Mucic Acid+Sebacic Acid+D-glucosaminic Acid, Mucic Acid+Sebacic Acid+Glycyl-L-Aspartic Acid, Mucic Acid+2,3-Butanediol+N-Acetyl-D-Galactosamine, Mucic Acid+3-Hydroxy-2-Butanone+N-Acetyl-D-Galactosamine, Glyocylic Acid+Inulin+Glycyl-L-Aspartic Acid, Glyocylic Acid+3-Methyl Glucose+D-glucosaminic Acid, Glyocylic Acid+3-Methyl Glucose+Glycyl-L-Aspartic Acid, Glyocylic Acid+Oxalic Acid+L-Alanine, Glyocylic Acid+Sebacic Acid+D-glucosaminic Acid, Glyocylic Acid+Sebacic Acid+Glycyl-L-Aspartic Acid, Glyocylic Acid+2,3-Butanediol+N-Acetyl-D-Galactosamine, Glyocylic Acid+3-Hydroxy-2-Butanone+N-Acetyl-D-Galactosamine, Phenylethyl-amine+3-Methyl Glucose+D-glucosaminic Acid, Phenylethyl-amine+3-Methyl Glucose+N-Acetyl-D-Galactosamine, Phenylethyl-amine+3-Methyl Glucose+Glycyl-L-Aspartic Acid, Phenylethyl-amine+Oxalic Acid+L-Alanine, Phenylethyl-amine+Oxalic Acid+N-Acetyl-D-Galactosamine, Phenylethyl-amine+Pectin+N-Acetyl-D-Galactosamine, D-Alanine+3-Methyl Glucose, D-Alanine+Oxalic Acid, L-Alanyl-Glycine+γ-Cyclodextrin, L-Alanyl-Glycine+Inulin, L-Alanyl-Glycine+Sebacic Acid, L-Alanyl-Glycine+2,3-Butanediol, L-Alanyl-Glycine+3-Hydroxy 2-Butanone, D-Saccharic Acid+Oxalic Acid+L-Alanine, Succinic Acid+Oxalic Acid+L-Alanine, Succinic Acid+Pectin+L-Alanine, Tween 40+Oxalic Acid+L-Alanine, Tween 40+Pectin+L-Alanine, α-Keto-Glutaric Acid+Inulin+Glycyl-L-Aspartic Acid, α-Keto-Glutaric Acid+Sebacic Acid+Glycyl-L-Aspartic Acid, α-Keto-Glutaric Acid+3-Hydroxy-2-Butanone+Glycyl-L-Aspartic Acid, m-Tartaric Acid+Oxalic Acid+L-Alanine, m-Tartaric Acid+Pectin+L-Alanine, Bromo Succinic Acid+γ-Cyclodextrin+D-glucosaminic Acid, Bromo Succinic Acid+γ-Cyclodextrin+Glycyl-L-Aspartic Acid, Bromo Succinic Acid+Inulin+D-glucosaminic Acid, Bromo Succinic Acid+Inulin+Glycyl-L-Aspartic Acid, Bromo Succinic Acid+3-Methyl Glucose+D-glucosaminic Acid, Bromo Succinic Acid+3-Methyl Glucose+Glycyl-L-Aspartic Acid, Bromo Succinic Acid+Sebacic Acid+D-glucosaminic Acid, Bromo Succinic Acid+Sebacic Acid+glycyl-L-Aspartic Acid, Bromo Succinic Acid+2,3-Butanediol+D-glucosaminic Acid, Bromo Succinic Acid+3-Hydroxy-2-Butanone+D-glucosaminic Acid, Bromo Succinic Acid+Pectin+D-Glucosaminic Acid, Bromo Succinic Acid+Pectin+Glycyl-L-Aspartic Acid, Phenylethyl-amine+a-Cyclodextrin+N-Acetyl-D-Galactosamine, Phenylethyl-amine+Turanose+N-Acetyl-D-Galactosamine, Phenylethyl-amine+L-Homoserine+N-Acetyl-D-Galactosamine, Phenylethyl-amine+2,3-Butanediol+N-Acetyl-D-Galactosamine, and Phenylethyl-amine+3-Hydroxy-2-Butanone+N-Acetyl-D-Galactosamine.

Embodiment 19: The method of embodiment 17, wherein the skin microbiota balancing agent is configured to provide the first desired ratio of Corynebacterium to Staphylococcus, the second desired ratio of Corynebacterium to Micrococcus, and the third desired ratio of Staphylococcus to Micrococcus.

Embodiment 20: The method of embodiment 19, wherein the at least one combination of carbohydrate sources is selected from the group consisting of: D-Alanine+3-Methyl Glucose, D-Alanine+Oxalic Acid, L-Alanyl-Glycine+y-Cyclodextrin, L-Alanyl-Glycine+Inulin, L-Alanyl-Glycine+Sebacic Acid, L-Alanyl-Glycine+2,3-Butanediol, L-Alanyl-Glycine+3-Hydroxy 2-Butanone, D-Saccharic Acid+Oxalic Acid+L-Alanine, Succinic Acid+Oxalic Acid+L-Alanine, Succinic Acid+Pectin+L-Alanine, Tween 40+Oxalic Acid+L-Alanine, Tween 40+Pectin+L-Alanine, α-Keto-Glutaric Acid+Inulin+Glycyl-L-Aspartic Acid, α-Keto-Glutaric Acid+Sebacic Acid+Glycyl-L-Aspartic Acid, α-Keto-Glutaric Acid+3-Hydroxy-2-Butanone+Glycyl-L-Aspartic Acid, m-Tartaric Acid+Oxalic Acid+L-Alanine, m-Tartaric Acid+Pectin+L-Alanine, Bromo Succinic Acid+γ-Cyclodextrin+D-glucosaminic Acid, Bromo Succinic Acid+γ-Cyclodextrin+Glycyl-L-Aspartic Acid, Bromo Succinic Acid+Inulin+D-glucosaminic Acid, Bromo Succinic Acid+Inulin+Glycyl-L-Aspartic Acid, Bromo Succinic Acid+3-Methyl Glucose+D-glucosaminic Acid, Bromo Succinic Acid+3-Methyl Glucose+Glycyl-L-Aspartic Acid, Bromo Succinic Acid+Sebacic Acid+D-glucosaminic Acid, Bromo Succinic Acid+Sebacic Acid+glycyl-L-Aspartic Acid, Bromo Succinic Acid+2,3-Butanediol+D-glucosaminic Acid, Bromo Succinic Acid+3-Hydroxy-2-Butanone+D-glucosaminic Acid, Bromo Succinic Acid+Pectin+D-Glucosaminic Acid, Bromo Succinic Acid+Pectin+Glycyl-L-Aspartic Acid, Phenylethyl-amine+α-Cyclodextrin+N-Acetyl-D-Galactosamine, Phenylethyl-amine+Turanose+N-Acetyl-D-Galactosamine, Phenylethyl-amine+L-Homoserine+N-Acetyl-D-Galactosamine, Phenylethyl-amine+2,3-Butanediol+N-Acetyl-D-Galactosamine, and Phenylethyl-amine+3-Hydroxy-2-Butanone+N-Acetyl-D-Galactosamine.

1. A composition for providing or maintaining a healthy skin microbiota, the composition comprising:
a carrier; and
a skin microbiota balancing agent, the skin microbiota balancing agent including at least one combination of carbohydrate sources including a first carbohydrate source and a second carbohydrate source, wherein the skin microbiota balancing agent is configured to provide at least two of the following desired ratios: a first desired ratio of Corynebacterium to Staphylococcus, a second desired ratio of Corynebacterium to Micrococcus, and a third desired ratio of Staphylococcus to Micrococcus, wherein the first desired ratio of Corynebacterium to Staphylococcus is 1.3, the second desired ratio of Corynebacterium to Micrococcus is 1.4, and the third desired ratio of Staphylococcus to Micrococcus is 1.1.

2. (canceled)

3. The composition of claim 1, wherein the at least one combination of carbohydrate sources is selected from the group consisting of: D-Alanine+γ-Cyclodextrin, D-Alanine+Sebacic Acid, D-Alanine+2,3-Butanediol, D-Aspartic Acid+Oxalic Acid, D-Aspartic Acid+Pectin, D-Threonine+3-Methyl Glucose, D-Threonine+Pectin, Fumaric Acid+α-Cyclodextrin, Fumaric Acid+Mannan, L-Alanyl-Glycine+3-Methyl Glucose, L-Alanyl-Glycine+Oxalic Acid, L-Alanyl-Glycine+Pectin, L-Malic Acid+Mannan, D-Saccharic Acid+Pectin+L-Alanine, Succinic Acid+3-Methyl Glucose+D-Glucosaminic Acid, Succinic Acid+3-Methyl Glucose+L-Alanine, Succinic Acid+3-Methyl Glucose+Glycyl-L-Aspartic Acid, Succinic Acid+Pectin+D-Glucosaminic Acid, Succinic Acid+Pectin+Glycyl-L-Aspartic Acid, Tween 40+3-Methyl Glucose+D-glucosaminic Acid, Tween 40+3-Methyl Glucose+L-Alanine, Tween 40+3-Methyl Glucose+Glycyl-L-Aspartic Acid, Tween 40+Pectin+D-glucosaminic Acid, Tween 40+Pectin+Glycyl-L-Aspartic Acid, α-Keto-Glutaric Acid+γ-Cyclodextrin+Glycyl-L-Aspartic Acid, α-Keto-Glutaric Acid+Inulin+D-glucosaminic Acid, α-Keto-Glutaric Acid+3-Methyl Glucose+Glycyl-L-Aspartic Acid, α-Keto-Glutaric Acid+γ-Amino Butyric Acid+Glycyl-L-Aspartic Acid, α-Keto-Glutaric Acid+Sebacic Acid+D-glucosaminic Acid, α-Keto-Glutaric Acid+2,3-Butanediol+Glycyl-L-Aspartic Acid, α-Keto-Glutaric Acid+3-Hydroxy-2-Butanone+D-glucosaminic Acid, α-Keto-Butyric Acid+α-Cyclodextrin+N-Acetyl-D-Galactosamine, α-Keto-Butyric Acid+Inulin+D-glucosaminic Acid, α-Keto-Butyric Acid+Inulin+Glycyl-L-Aspartic Acid, α-Keto-Butyric Acid+3-Methyl Glucose+D-glucosaminic Acid, α-Keto-Butyric Acid+3-Methyl Glucose+Glycyl-L-Aspartic Acid, α-Keto-Butyric Acid+Oxalic Acid+L-Alanine, α-Keto-Butyric Acid+Sebacic Acid+D-glucosaminic Acid, α-Keto-Butyric Acid+Sebacic Acid+Glycyl-L-Aspartic Acid, α-Keto-Butyric Acid+L-Homoserine+N-Acetyl-D-Galactosamine, α-Keto-Butyric Acid+2,3-Butanediol+N-Acetyl-D-Galactosamine, α-Keto-Butyric Acid+3-Hydroxy-2-Butanone+N-Acetyl-D-Galactosamine, m-Tartaric Acid+Pectin+D-Glucosaminic Acid, m-Tartaric Acid+Pectin+Glycyl-L-Aspartic Acid, α-Hydroxy Butyric Acid+Inulin+D-glucosaminic Acid, α-Hydroxy Butyric Acid+Inulin+Glycyl-L-Aspartic Acid, α-Hydroxy Butyric Acid+3-Methyl Glucose+D-glucosaminic Acid, α-Hydroxy Butyric Acid+3-Methyl Glucose+Glycyl-L-Aspartic Acid, α-Hydroxy Butyric Acid+Oxalic Acid+L-Alanine, α-Hydroxy Butyric Acid+Sebacic Acid+D-glucosaminic Acid, α-Hydroxy Butyric Acid+Sebacic Acid+Glycyl-L-Aspartic Acid, α-Hydroxy Butyric Acid+2,3-Butanediol+N-Acetyl-D-Galactosamine, α-Hydroxy Butyric Acid+3-Hydroxy-2-Butanone+N-Acetyl-D-Galactosamine, Citric Acid+α-Cyclodextrin+Glycyl-L-Glutamic Acid, Citric Acid+α-Cyclodextrin+Tricarballylic Acid, Citric Acid+Mannan+Glycyl-L-Glutamic Acid, Citric Acid+Mannan+Tricarballylic Acid, Citric Acid+Oxalic Acid+L-Alanine, Citric Acid+Pectin+L-Alanine, Bromo Succinic Acid+2,3-Butanediol+N-Acetyl-D-Galactosamine, Bromo Succinic Acid+2,3-Butanediol+glycyl-L-Aspartic Acid, Bromo Succinic Acid+3-Hydroxy-2-Butanone+N-Acetyl-D-Galactosamine, Bromo Succinic Acid+3-Hydroxy-2-Butanone+Glycyl-L-Aspartic Acid, Bromo Succinic Acid+Pectin+L-Alanine, Mucic Acid+Inulin+D-glucosaminic Acid, Mucic Acid+Inulin+Glycyl-L-Aspartic Acid, Mucic Acid+3-Methyl Glucose+D-glucosaminic Acid, Mucic Acid+3-Methyl Glucose+Glycyl-L-Aspartic Acid, Mucic Acid+Oxalic Acid+L-Alanine, Mucic Acid+Sebacic Acid+D-glucosaminic Acid, Mucic Acid+Sebacic Acid+Glycyl-L-Aspartic Acid, Mucic Acid+2,3-Butanediol+N-Acetyl-D-Galactosamine, Mucic Acid+3-Hydroxy-2-Butanone+N-Acetyl-D-Galactosamine, Glyocylic Acid+Inulin+Glycyl-L-Aspartic Acid, Glyocylic Acid+3-Methyl Glucose+D-glucosaminic Acid, Glyocylic Acid+3-Methyl Glucose+Glycyl-L-Aspartic Acid, Glyocylic Acid+Oxalic Acid+L-Alanine, Glyocylic Acid+Sebacic Acid+D-glucosaminic Acid, Glyocylic Acid+Sebacic Acid+Glycyl-L-Aspartic Acid, Glyocylic Acid+2,3-Butanediol+N-Acetyl-D-Galactosamine, Glyocylic Acid+3-Hydroxy-2-Butanone+N-Acetyl-D-Galactosamine, Phenylethylamine+3-Methyl Glucose+D-glucosaminic Acid, Phenylethyl-amine+3-Methyl Glucose+N-Acetyl-D-Galactosamine, Phenylethyl-amine+3-Methyl Glucose+Glycyl-L-Aspartic Acid, Phenylethyl-amine+Oxalic Acid+L-Alanine, Phenylethyl-amine+Oxalic Acid+N-Acetyl-D-Galactosamine, Phenylethylamine+Pectin+N-Acetyl-D-Galactosamine, D-Alanine+3-Methyl Glucose, D-Alanine+Oxalic Acid, L-Alanyl-Glycine+γ-Cyclodextrin, L-Alanyl-Glycine+Inulin, L-Alanyl-Glycine+Sebacic Acid, L-Alanyl-Glycine+2,3-Butanediol, L-Alanyl-Glycine+3-Hydroxy 2-Butanone, D-Saccharic Acid+Oxalic Acid+L-Alanine, Succinic Acid+Oxalic Acid+L-Alanine, Succinic Acid+Pectin+L-Alanine, Tween 40+Oxalic Acid+L-Alanine, Tween 40+Pectin+L-Alanine, α-Keto-Glutaric Acid+Inulin+Glycyl-L-Aspartic Acid, α-Keto-Glutaric Acid+Sebacic Acid+Glycyl-L-Aspartic Acid, α-Keto-Glutaric Acid+3-Hydroxy-2-Butanone+Glycyl-L-Aspartic Acid, m-Tartaric Acid+Oxalic Acid+L-Alanine, m-Tartaric Acid+Pectin+L-Alanine, Bromo Succinic Acid+y-Cyclodextrin+D-glucosaminic Acid, Bromo Succinic Acid+γ-Cyclodextrin+Glycyl-L-Aspartic Acid, Bromo Succinic Acid+Inulin+D-glucosaminic Acid, Bromo Succinic Acid+Inulin+Glycyl-L-Aspartic Acid, Bromo Succinic Acid+3-Methyl Glucose+D-glucosaminic Acid, Bromo Succinic Acid+3-Methyl Glucose+Glycyl-L-Aspartic Acid, Bromo Succinic Acid+Sebacic Acid+D-glucosaminic Acid, Bromo Succinic Acid+Sebacic Acid+glycyl-L-Aspartic Acid, Bromo Succinic Acid+2,3-Butanediol+D-glucosaminic Acid, Bromo Succinic Acid+3-Hydroxy-2-Butanone+D-glucosaminic Acid, Bromo Succinic Acid+Pectin+D-Glucosaminic Acid, Bromo Succinic Acid+Pectin+Glycyl-L-Aspartic Acid, Phenylethyl-amine+α-Cyclodextrin+N-Acetyl-D-Galactosamine, Phenylethyl-amine+Turanose+N-Acetyl-D-Galactosamine, Phenylethyl-amine+L-Homoserine+N-Acetyl-D-Galactosamine, Phenylethyl-amine+2,3-Butanediol+N-Acetyl-D-Galactosamine, and Phenylethyl-amine+3-Hydroxy-2-Butanone+N-Acetyl-D-Galactosamine.

4. The composition of claim 1, wherein the skin microbiota balancing agent is configured to provide the first desired ratio of Corynebacterium to Staphylococcus, the second desired ratio of Corynebacterium to Micrococcus, and the third desired ratio of Staphylococcus to Micrococcus.

5. The composition of claim 4, wherein the at least one combination of carbohydrate sources is selected from the group consisting of: D-Alanine+3-Methyl Glucose, D-Alanine+Oxalic Acid, L-Alanyl-Glycine+γ-Cyclodextrin, L-Alanyl-Glycine+Inulin, L-Alanyl-Glycine+Sebacic Acid, L-Alanyl-Glycine+2,3-Butanediol, L-Alanyl-Glycine+3-Hydroxy 2-Butanone, D-Saccharic Acid+Oxalic Acid+L-Alanine, Succinic Acid+Oxalic Acid+L-Alanine, Succinic Acid+Pectin+L-Alanine, Tween 40 +Oxalic Acid+L-Alanine, Tween 40+Pectin+L-Alanine, α-Keto-Glutaric Acid+Inulin+Glycyl-L-Aspartic Acid, α-Keto-Glutaric Acid+Sebacic Acid+Glycyl-L-Aspartic Acid, α-Keto-Glutaric Acid+3-Hydroxy-2-Butanone+Glycyl-L-Aspartic Acid, m-Tartaric Acid+Oxalic Acid+L-Alanine, m-Tartaric Acid+Pectin+L-Alanine, Bromo Succinic Acid+y-Cyclodextrin+D-glucosaminic Acid, Bromo Succinic Acid+γ-Cyclodextrin+Glycyl-L-Aspartic Acid, Bromo Succinic Acid+Inulin+D-glucosaminic Acid, Bromo Succinic Acid+Inulin+Glycyl-L-Aspartic Acid, Bromo Succinic Acid+3-Methyl Glucose+D-glucosaminic Acid, Bromo Succinic Acid+3-Methyl Glucose+Glycyl-L-Aspartic Acid, Bromo Succinic Acid+Sebacic Acid+D-glucosaminic Acid, Bromo Succinic Acid+Sebacic Acid+glycyl-L-Aspartic Acid, Bromo Succinic Acid+2,3-Butanediol+D-glucosaminic Acid, Bromo Succinic Acid+3-Hydroxy-2-Butanone+D-glucosaminic Acid, Bromo Succinic Acid+Pectin+D-Glucosaminic Acid, Bromo Succinic Acid+Pectin+Glycyl-L-Aspartic Acid, Phenylethyl-amine+α-Cyclodextrin+N-Acetyl-D-Galactosamine, Phenylethyl-amine+Turanose+N-Acetyl-D-Galactosamine, Phenylethyl-amine+L-Homoserine+N-Acetyl-D-Galactosamine, Phenylethyl-amine+2,3-Butanediol+N-Acetyl-D-Galactosamine, and Phenylethyl-amine+3-Hydroxy-2-Butanone+N-Acetyl-D-Galactosamine.

6. The composition of claim 1, wherein the composition is applied to a component of an absorbent article or to a wipe.

7. The composition of claim 1, wherein the composition is in the form of a liquid or cream.

8. A composition for providing or maintaining a healthy skin microbiota, the composition comprising:
a carrier; and
a skin microbiota balancing agent, the skin microbiota balancing agent including at least one combination of carbohydrate sources, the combination of carbohydrate sources being selected from the group consisting of: D-Alanine+3-Methyl Glucose, D-Alanine+Oxalic Acid, L-Alanyl-Glycine+γ-Cyclodextrin, L-Alanyl-Glycine+Inulin, L-Alanyl-Glycine+Sebacic Acid, L-Alanyl-Glycine+2,3-Butanediol, L-Alanyl-Glycine+3-Hydroxy 2-Butanone, D-Saccharic Acid+Oxalic Acid+L-Alanine, Succinic Acid+Oxalic Acid+L-Alanine, Succinic Acid+Pectin+L-Alanine, Tween 40+Oxalic Acid+L-Alanine, Tween 40+Pectin+L-Alanine, α-Keto-Glutaric Acid+Inulin+Glycyl-L-Aspartic Acid, α-Keto-Glutaric Acid+Sebacic Acid+Glycyl-L-Aspartic Acid, α-Keto-Glutaric Acid+3-Hydroxy-2-Butanone+Glycyl-L-Aspartic Acid, m-Tartaric Acid+Oxalic Acid+L-Alanine, m-Tartaric Acid+Pectin+L-Alanine, Bromo Succinic Acid+y-Cyclodextrin+D-glucosaminic Acid, Bromo Succinic Acid+γ-Cyclodextrin+Glycyl-L-Aspartic Acid, Bromo Succinic Acid+Inulin+D-glucosaminic Acid, Bromo Succinic Acid+Inulin+Glycyl-L-Aspartic Acid, Bromo Succinic Acid+3-Methyl Glucose+D-glucosaminic Acid, Bromo Succinic Acid+3-Methyl Glucose+Glycyl-L-Aspartic Acid, Bromo Succinic Acid+Sebacic Acid+D-glucosaminic Acid, Bromo Succinic Acid+Sebacic Acid+glycyl-L-Aspartic Acid, Bromo Succinic Acid+2,3-Butanediol+D-glucosaminic Acid, Bromo Succinic Acid+3-Hydroxy-2-Butanone+D-glucosaminic Acid, Bromo Succinic Acid+Pectin+D-Glucosaminic Acid, Bromo Succinic Acid+Pectin+Glycyl-L-Aspartic Acid, Phenylethyl-amine+α-Cyclodextrin+N-Acetyl-D-Galactosamine, Phenylethyl-amine+Turanose+N-Acetyl-D-Galactosamine, Phenylethyl-amine+L-Homoserine+N-Acetyl-D-Galactosamine, Phenylethyl-amine+2,3-Butanediol+N-Acetyl-D-Galactosamine, and Phenylethyl-amine+3-Hydroxy-2-Butanone+N-Acetyl-D-Galactosamine.

9. The composition of claim 8, the skin microbiota balancing agent is configured to provide a first desired ratio of Corynebacterium to Staphylococcus, a second desired ratio of Corynebacterium to Micrococcus, and a third desired ratio of Staphylococcus to Micrococcus.

10. The composition of claim 9, wherein the first desired ratio of Corynebacterium to Staphylococcus is 1.3, the second desired ratio of Corynebacterium to Micrococcus is 1.4, and the third desired ratio of Staphylococcus to Micrococcus is 1.1.

11. The composition of claim 8, wherein the wherein the composition is applied to a component of an absorbent article or to a wipe.

12. The composition of claim 8, wherein the composition is in the form of a liquid or cream.

13. A method to provide or maintain a healthy skin microbiota on a subject, the method comprising:
creating a prebiotic composition configured to maintain at least one of a first desired ratio of Corynebacterium to Staphylococcus, a second desired ratio of Corynebacterium to Micrococcus, and a third desired ratio of Staphylococcus to Micrococcus, wherein the first desired ratio of Corynebacterium to Staphylococcus is 1.3, the second desired ratio of Corynebacterium to Micrococcus is 1.4, and the third desired ratio of Staphylococcus to Micrococcus is 1.1, the prebiotic composition comprising:
a carrier; and
a skin microbiota balancing agent comprising at least a first carbohydrate source, selecting the first carbohydrate source to maintain the at least one of the first desired ratio of Corynebacterium to Staphylococcus, the second desired ratio of Corynebacterium to Micrococcus, and the third desired ratio of Staphylococcus to Micrococcus; and
providing instruction to apply the prebiotic composition to the subject.

14. The method of claim 13, further comprising: administering the prebiotic composition to the subject to provide or maintain a healthy skin microbiota in the subject.

15. (canceled)

16. The method of claim 13, wherein the prebiotic composition is configured to maintain at least two of the first desired ratio of Corynebacterium to Staphylococcus, the second desired ratio of Corynebacterium to Micrococcus, and the third desired ratio of Staphylococcus to Micrococcus.

17. The method of claim 16, wherein the skin microbiota balancing agent includes at least one combination of carbohydrate sources, the at least one combination of carbohydrate sources including the first carbohydrate source and a second carbohydrate source.

18. The method of claim 17, wherein the at least one combination of carbohydrate sources is selected from the group consisting of: D-Alanine+γ-Cyclodextrin, D-Alanine+Sebacic Acid, D-Alanine+2,3-Butanediol, D-Aspartic Acid+Oxalic Acid, D-Aspartic Acid+Pectin, D-Threonine+3-Methyl Glucose, D-Threonine+Pectin, Fumaric Acid+α-Cyclodextrin, Fumaric Acid+Mannan, L-Alanyl-Glycine+3-Methyl Glucose, L-Alanyl-Glycine+Oxalic Acid, L-Alanyl-Glycine+Pectin, L-Malic Acid+Mannan, D-Saccharic Acid+Pectin+L-Alanine, Succinic Acid+3-Methyl Glucose+D-Glucosaminic Acid, Succinic Acid+3-Methyl Glucose+L-Alanine, Succinic Acid+3-Methyl Glucose+Glycyl-L-Aspartic Acid, Succinic Acid+Pectin+D-Glucosaminic Acid, Succinic Acid+Pectin+Glycyl-L-Aspartic Acid, Tween 40+3-Methyl Glucose+D-glucosaminic Acid, Tween 40+3-Methyl Glucose+L-Alanine, Tween 40+3-Methyl Glucose+Glycyl-L-Aspartic Acid, Tween 40+Pectin+D-glucosaminic Acid, Tween 40+Pectin+Glycyl-L-Aspartic Acid, α-Keto-Glutaric Acid+γ-Cyclodextrin+Glycyl-L-Aspartic Acid, α-Keto-Glutaric Acid+Inulin+D-glucosaminic Acid, α-Keto-Glutaric Acid+3-Methyl Glucose+Glycyl-L-Aspartic Acid, α-Keto-Glutaric Acid+γ-Amino Butyric Acid+Glycyl-L-Aspartic Acid, α-Keto-Glutaric Acid+Sebacic Acid+D-glucosaminic Acid, α-Keto-Glutaric Acid+2,3-Butanediol+Glycyl-L-Aspartic Acid, α-Keto-Glutaric Acid+3-Hydroxy-2-Butanone+D-glucosaminic Acid, α-Keto-Butyric Acid+α-Cyclodextrin+N-Acetyl-D-Galactosamine, α-Keto-Butyric Acid+Inulin+D-glucosaminic Acid, α-Keto-Butyric Acid+Inulin+Glycyl-L-Aspartic Acid, α-Keto-Butyric Acid+3-Methyl Glucose+D-glucosaminic Acid, α-Keto-Butyric Acid+3-Methyl Glucose+Glycyl-L-Aspartic Acid, α-Keto-Butyric Acid+Oxalic Acid+L-Alanine, α-Keto-Butyric Acid+Sebacic Acid+D-glucosaminic Acid, α-Keto-Butyric Acid+Sebacic Acid+Glycyl-L-Aspartic Acid, α-Keto-Butyric Acid+L-Homoserine+N-Acetyl-D-Galactosamine, α-Keto-Butyric Acid+2,3-Butanediol+N-Acetyl-D-Galactosamine, α-Keto-Butyric Acid+3-Hydroxy-2-Butanone+N-Acetyl-D-Galactosamine, m-Tartaric Acid+Pectin+D-Glucosaminic Acid, m-Tartaric Acid+Pectin+Glycyl-L-Aspartic Acid, α-Hydroxy Butyric Acid+Inulin+D-glucosaminic Acid, α-Hydroxy Butyric Acid+Inulin+Glycyl-L-Aspartic Acid, α-Hydroxy Butyric Acid+3-Methyl Glucose+D-glucosaminic Acid, α-Hydroxy Butyric Acid+3-Methyl Glucose+Glycyl-L-Aspartic Acid, α-Hydroxy Butyric Acid+Oxalic Acid+L-Alanine, α-Hydroxy Butyric Acid+Sebacic Acid+D-glucosaminic Acid, α-Hydroxy Butyric Acid+Sebacic Acid+Glycyl-L-Aspartic Acid, α-Hydroxy Butyric Acid+2,3-Butanediol+N-Acetyl-D-Galactosamine, α-Hydroxy Butyric Acid+3-Hydroxy-2-Butanone+N-Acetyl-D-Galactosamine, Citric Acid+α-Cyclodextrin+Glycyl-L-Glutamic Acid, Citric Acid+α-Cyclodextrin+Tricarballylic Acid, Citric Acid+Mannan+Glycyl-L-Glutamic Acid, Citric Acid+Mannan+Tricarballylic Acid, Citric Acid+Oxalic Acid+L-Alanine, Citric Acid+Pectin+L-Alanine, Bromo Succinic Acid+2,3-Butanediol+N-Acetyl-D-Galactosamine, Bromo Succinic Acid+2,3-Butanediol+glycyl-L-Aspartic Acid, Bromo Succinic Acid+3-Hydroxy-2-Butanone+N-Acetyl-D-Galactosamine, Bromo Succinic Acid+3-Hydroxy-2-Butanone+Glycyl-L-Aspartic Acid, Bromo Succinic Acid+Pectin+L-Alanine, Mucic Acid+Inulin+D-glucosaminic Acid, Mucic Acid+Inulin+Glycyl-L-Aspartic Acid, Mucic Acid+3-Methyl Glucose+D-glucosaminic Acid, Mucic Acid+3-Methyl Glucose+Glycyl-L-Aspartic Acid, Mucic Acid+Oxalic Acid+L-Alanine, Mucic Acid+Sebacic Acid+D-glucosaminic Acid, Mucic Acid+Sebacic Acid+Glycyl-L-Aspartic Acid, Mucic Acid+2,3-Butanediol+N-Acetyl-D-Galactosamine, Mucic Acid+3-Hydroxy-2-Butanone+N-Acetyl-D-Galactosamine, Glyocylic Acid+Inulin+Glycyl-L-Aspartic Acid, Glyocylic Acid+3-Methyl Glucose+D-glucosaminic Acid, Glyocylic Acid+3-Methyl Glucose+Glycyl-L-Aspartic Acid, Glyocylic Acid+Oxalic Acid+L-Alanine, Glyocylic Acid+Sebacic Acid+D-glucosaminic Acid, Glyocylic Acid+Sebacic Acid+Glycyl-L-Aspartic Acid, Glyocylic Acid+2,3-Butanediol+N-Acetyl-D-Galactosamine, Glyocylic Acid+3-Hydroxy-2-Butanone+N-Acetyl-D-Galactosamine, Phenylethyl-amine+3-Methyl Glucose+D-glucosaminic Acid, Phenylethyl-amine+3-Methyl Glucose+N-Acetyl-D-Galactosamine, Phenylethyl-amine+3-Methyl Glucose+Glycyl-L-Aspartic Acid, Phenylethyl-amine+Oxalic Acid+L-Alanine, Phenylethyl-amine+Oxalic Acid+N-Acetyl-D-Galactosamine, Phenylethyl-amine+Pectin+N-Acetyl-D-Galactosamine, D-Alanine+3-Methyl Glucose, D-Alanine+Oxalic Acid, L-Alanyl-Glycine+γ-Cyclodextrin, L-Alanyl-Glycine+Inulin, L-Alanyl-Glycine+Sebacic Acid, L-Alanyl-Glycine+2,3-Butanediol, L-Alanyl-Glycine+3-Hydroxy 2-Butanone, D-Saccharic Acid+Oxalic Acid+L-Alanine, Succinic Acid+Oxalic Acid+L-Alanine, Succinic Acid+Pectin+L-Alanine, Tween 40+Oxalic Acid+L-Alanine, Tween 40+Pectin+L-Alanine, α-Keto-Glutaric Acid+Inulin+Glycyl-L-Aspartic Acid, α-Keto-Glutaric Acid+Sebacic Acid+Glycyl-L-Aspartic Acid, α-Keto-Glutaric Acid+3-Hydroxy-2-Butanone+Glycyl-L-Aspartic Acid, m-Tartaric Acid+Oxalic Acid+L-Alanine, m-Tartaric Acid+Pectin+L-Alanine, Bromo Succinic Acid+y-Cyclodextrin+D-glucosaminic Acid, Bromo Succinic Acid+y-Cyclodextrin+Glycyl-L-Aspartic Acid, Bromo Succinic Acid+Inulin+D-glucosaminic Acid, Bromo Succinic Acid+Inulin+Glycyl-L-Aspartic Acid, Bromo Succinic Acid+3-Methyl Glucose+D-glucosaminic Acid, Bromo Succinic Acid+3-Methyl Glucose+Glycyl-L-Aspartic Acid, Bromo Succinic Acid+Sebacic Acid+D-glucosaminic Acid, Bromo Succinic Acid+Sebacic Acid+glycyl-L-Aspartic Acid, Bromo Succinic Acid+2,3-Butanediol+D-glucosaminic Acid, Bromo Succinic Acid+3-Hydroxy-2-Butanone+D-glucosaminic Acid, Bromo Succinic Acid+Pectin+D-Glucosaminic Acid, Bromo Succinic Acid+Pectin+Glycyl-L-Aspartic Acid, Phenylethyl-amine+α-Cyclodextrin+N-Acetyl-D-Galactosamine, Phenylethyl-amine+Turanose+N-Acetyl-D-Galactosamine, Phenylethyl-amine+L-Homoserine+N-Acetyl-D-Galactosamine, Phenylethyl-amine+2,3-Butanediol+N-Acetyl-D-Galactosamine, and Phenylethyl-amine+3-Hydroxy-2-Butanone+N-Acetyl-D-Galactosamine.

19. The method of claim 17, wherein the skin microbiota balancing agent is configured to provide the first desired ratio of Corynebacterium to Staphylococcus, the second desired ratio of Corynebacterium to Micrococcus, and the third desired ratio of Staphylococcus to Micrococcus.

20. The method of claim 19, wherein the at least one combination of carbohydrate sources is selected from the group consisting of: D-Alanine+3-Methyl Glucose, D-Alanine+Oxalic Acid, L-Alanyl-Glycine+γ-Cyclodextrin, L-Alanyl-Glycine+Inulin, L-Alanyl-Glycine+Sebacic Acid, L-Alanyl-Glycine+2,3-Butanediol, L-Alanyl-Glycine+3-Hydroxy 2-Butanone, D-Saccharic Acid+Oxalic Acid+L-Alanine, Succinic Acid+Oxalic Acid+L-Alanine, Succinic Acid+Pectin+L-Alanine, Tween 40+Oxalic Acid+L-Alanine, Tween 40+Pectin+L-Alanine, α-Keto-Glutaric Acid+Inulin+Glycyl-L-Aspartic Acid, α-Keto-Glutaric Acid+Sebacic Acid+Glycyl-L-Aspartic Acid, α-Keto-Glutaric Acid+3-Hydroxy-2-Butanone+Glycyl-L-Aspartic Acid, m-Tartaric Acid+Oxalic Acid+L-Alanine, m-Tartaric Acid+Pectin+L-Alanine, Bromo Succinic Acid+γ-Cyclodextrin+D-glucosaminic Acid, Bromo Succinic Acid+γ-Cyclodextrin+Glycyl-L-Aspartic Acid, Bromo Succinic Acid+Inulin+D-glucosaminic Acid, Bromo Succinic Acid+Inulin+Glycyl-L-Aspartic Acid, Bromo Succinic Acid+3-Methyl Glucose+D-glucosaminic Acid, Bromo Succinic Acid+3-Methyl Glucose+Glycyl-L-Aspartic Acid, Bromo Succinic Acid+Sebacic Acid+D-glucosaminic Acid, Bromo Succinic Acid+Sebacic Acid+glycyl-L-Aspartic Acid, Bromo Succinic Acid+2,3-Butanediol+D-glucosaminic Acid, Bromo Succinic Acid+3-Hydroxy-2-Butanone+D-glucosaminic Acid, Bromo Succinic Acid+Pectin+D-Glucosaminic Acid, Bromo Succinic Acid+Pectin+Glycyl-L-Aspartic Acid, Phenylethyl-amine+α-Cyclodextrin+N-Acetyl-D-Galactosamine, Phenylethyl-amine+Turanose+N-Acetyl-D-Galactosamine, Phenylethyl-amine+L-Homoserine+N-Acetyl-D-Galactosamine, Phenylethyl-amine+2,3-Butanediol+N-Acetyl-D-Galactosamine, and Phenylethyl-amine+3-Hydroxy-2-Butanone+N-Acetyl-D-Galactosamine.
